(19) United States
(12) Patent Application Publication
(10) Pub. No.: US 2010/0135939 A1
(43) Pub. Date: Jun. 3, 2010

(54) UV LIGHT-PROTECTIVE AGENT BASED ON MIXED INORGANIC-ORGANIC SYSTEMS

(75) Inventors: Stephan Lehmann, Limburgerhof (DE); Richard Riggs, Mannheim (DE); Valerie Andre, Ludwigshafen (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/594,207

(22) PCT Filed: Mar. 13, 2008
(86) PCT No.: PCT/EP08/53046

§ 371 Date: Oct. 1, 2009

(30) Foreign Application Priority Data
Apr. 4, 2007 (EP) 07105661.8

(51) Int. Cl. A61K 8/36; A61K 8/30; A61Q 17/04

(52) U.S. Cl. 424/60; 424/59

(57) ABSTRACT

The present invention relates to photoprotective preparations, in particular in the field of cosmetics, based on inorganic-organic mixed systems. The present invention relates in particular to photoprotective preparations comprising nanoparticles of at least one metal derivative, at least one photoprotective compound chemically bonded to these metal derivative nanoparticles, and at least one further organic UV photoprotective agent which is different from the photoprotective compound bonded to the metal derivative nanoparticles.

The present invention relates to photoprotective preparations, in particular in the field of cosmetics, based on inorganic-organic mixed systems. In particular, the present invention relates to photoprotective preparations comprising nanoparticles of at least one metal derivative, at least one photoprotective compound chemically bonded to these metal derivative nanoparticles and at least one further organic UV photoprotective agent which is different from the photoprotective compound bonded to the metal derivative nanoparticles.

Metal oxides are used for diverse purposes, thus, for example, as white pigment, as catalyst, as constituent of antibacterial skin-protection ointments and as activator for the vulcanization of rubber. Cosmetic sunscreen compositions comprise finely divided zinc oxide or titanium dioxide as UV-absorbing pigments.

Within the scope of the present application, the term “nanoparticles” is used to refer to particles with an average diameter of 2, preferably from 5 to 10 000 nm, determined by means of electron-microscopic methods.

WO 2007/017587 describes the cosmetic use of a composite material comprising a metal derivative and an organic UV photoprotective filter, where the UV photoprotective filter is bonded chemically to the metal derivative. Preferably, the chemical bond is a covalent bond. WO 2007/017586 describes a method of producing such composite materials.

It was an object of the present invention to provide photoprotective preparations which have an improved profile of properties compared with the photoprotective preparations known from the prior art.

This object was achieved by photoprotective preparations comprising
nanoparticles of at least one metal derivative
at least one structural unit chemically bonded to the nanoparticles of at least one metal derivative, selected from the group consisting of derivatives of triazine, derivatives of hydroxybenzophenone and derivatives of 4,4′diarylbutadiene.

The object was achieved in particular by photoprotective preparations comprising
nanoparticles of at least one metal derivative
at least one triazine derivative chemically bonded to the nanoparticles of at least one metal derivative.

The chemically bonded triazine derivatives are bonded chemically to the metal derivative nanoparticles, for example, via at least one —COO or one —SO₂O function. The —COO or —SO₂O structural elements can be obtained, for example, from the free acid or its salt. This is described, for example, in sections [0015] to [0018] of WO 2007/017586, to which reference is made here in its entirety.

Besides at least one —COO and/or —SO₂O group or group that can be converted into a —COO and/or —SO₂O group, suitable triazines comprise, for example, the following structural motif:

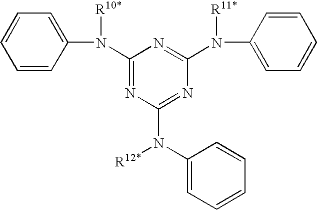

Thus, for example, it is possible to use the s-triazine derivatives described in the European laid-open specification EP 570 838 A1, the chemical structure of which is given by the generic formula

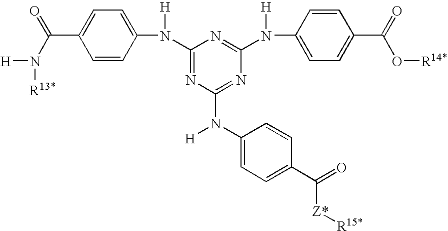

where
R¹³* is a branched or unbranched C₁-C₁₈-alkyl radical, a C₅-C₁₂-cycloalkyl radical, optionally substituted by one or more C₁-C₄-alkyl groups,
Z* is an oxygen atom or an NH group,
R¹⁴* is a branched or unbranched C₁-C₁₈-alkyl radical, a C₅-C₁₂-cycloalkyl radical, optionally substituted by one or more C₁-C₄-alkyl groups, or a hydrogen atom, an alkali metal atom, an ammonium group or a group of the formula

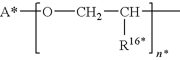

in which
A* is a branched or unbranched C₁-C₁₈-alkyl radical, a C₅-C₁₂-cycloalkyl or aryl radical, optionally substituted by one or more C₁-C₄-alkyl groups,
R¹⁶* is a hydrogen atom or a methyl group,
n* is a number from 1 to 10,
R¹⁵* is a branched or unbranched C₁-C₁₈-alkyl radical, a C₅-C₁₂-cycloalkyl radical, optionally substituted by one or more C₁-C₄-alkyl groups, if X is the NH group, and a branched or unbranched C₁-C₁₈-alkyl radical, a C₅-C₁₂-cycloalkyl radical, optionally substituted by one or more C₁-C₄-alkyl groups, or a hydrogen atom, an alkali metal atom, an ammonium group or a group of the formula

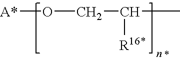

in which
A* is a branched or unbranched C₁-C₁₈-alkyl radical, a C₅-C₁₂-cycloalkyl or aryl radical, optionally substituted by one or more C₁-C₄-alkyl groups,
R¹⁶* is a hydrogen atom or a methyl group,
n* is a number from 1 to 10,
if X is an oxygen atom.

The abovementioned triazines are converted, by cleavage of at least one of the ester and/or amide bonds, into a form which is able to form a chemical, preferably covalent, bond to the metal derivative. The way in which the triazines are to be converted into the corresponding derivatives is known to the person skilled in the art.

For the purposes of the present invention, a preferred triazine derivative is also an asymmetrically substituted s-triazine, the chemical structure of which is given by the formula

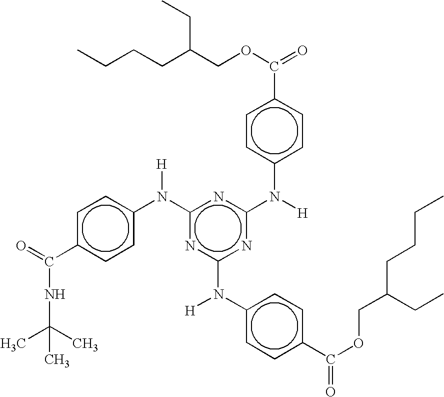

which is also referred to below as dioctylbutylamidotriazone (INCI: Diethylhexylbutamidotriazone) and is available under the trade name UVASORB® HEB from 3V Sigma. By cleaving at least one of the ester and/or amide bonds, this triazine is converted to a form which is then able to form a chemical, preferably covalent, bond to the metal derivative nanoparticle. It is preferred to cleave at least one of the ester bonds hydrolytically and, through the resulting free acid or salt, to render possible a bond to the metal derivative.

Also advantageous for the purposes of the present invention is a symmetrically substituted s-triazine, tris(2-ethylhexyl) 4,4′,4″-(1,3,5-triazine-2,4,6-triyltriimino)trisbenzoate, synonym: 2,4,6-tris[anilino(p-carbo-2′-ethyl-1′-hexyloxy)]-1,3,5-triazine (INCI: Ethylhexyl Triazone), which is sold by BASF Aktiengesellschaft under the trade name UVINUL® T 150. Through cleavage of at least one of the ester bonds, this triazine is converted to a form which is then able to form a chemical, preferably covalent, bond to the metal derivative.

The object underlying this invention was also achieved in particular by cosmetic preparations comprising
nanoparticles of at least one metal derivative
at least one hydroxybenzophenone derivative chemically bonded to the nanoparticles of at least one metal derivative.

A particularly advantageous hydroxybenzophenone for the purposes of the present invention is hexyl 2-(4′-(diethylamino)-2′-hydroxybenzoyl)benzoate (Uvinul®A Plus) of the following formula:

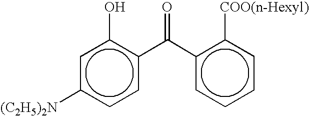

For bonding to the metal derivative nanoparticles, the hexyl ester is, for example, converted to the —COOH form or the salt form.

The object underlying this invention was also achieved in particular by cosmetic preparations comprising
nanoparticles of at least one metal derivative
at least one 4,4′-diarylbutadiene derivative chemically bonded to the nanoparticles of at least one metal derivative.

The 4,4-diarylbutadiene derivatives are described in tables 1, 2, 3 and 4 of EP-A 916 335, to which reference is hereby made. Of particular suitability here are the 4,4-diarylbutadiene derivatives specified in tables 1, 2 and 3. A particularly suitable 4,4′-diarylbutadiene is 1,1-[(2,2′-dimethylpropoxy)carbonyl]-4,4-diphenyl-1,3-butadiene.

It is particularly preferred if, besides the nanoparticles of at least one metal derivative, and besides the at least one organic UV photoprotective agent which is chemically bonded to the metal derivative nanoparticles, the preparations comprise at least one further organic UV photoprotective agent which is different from the photoprotective compound bonded to the metal derivative nanoparticles. Such further suitable UV photoprotective agents are specified in the table below, without being limited thereto:

| | | CAS No. |
|---|---|---|
| No. | Substance | (=acid) |
| 1 | 4-Aminobenzoic acid | 150-13-0 |
| 2 | 3-(4′-Trimethylammonium)benzylidenebornan-2-one methyl | 52793-97-2 |
| | sulfate | |
| 3 | 3,3,5-Trimethylcyclohexyl salicylate | 118-56-9 |
| | (homosalate) | |
| 4 | 2-Hydroxy-4-methoxybenzophenone (oxybenzone) | 131-57-7 |
| 5 | 2-Phenylbenzimidazole-5-sulfonic acid and its potassium, | 27503-81-7 |
| | sodium and triethanolamine salts | |
| 6 | 3,3′-(1,4-Phenylenedimethine)bis(7,7-dimethyl-2-oxo- | 90457-82-2 |
| | bicyclo[2.2.1]heptane-1-methanesulfonic acid) and its salts | |
| 7 | Polyethoxyethyl 4-bis(polyethoxy)aminobenzoate | 113010-52-9 |
| 8 | 2-Ethylhexyl 4-dimethylaminobenzoate | 21245-02-3 |
| 9 | 2-Ethylhexyl salicylate | 118-60-5 |
| 10 | 2-Isoamyl 4-methoxycinnamate | 71617-10-2 |
| 12 | 2-Hydroxy-4-methoxybenzophenone-5-sulfonic acid | 4065-45-6 |
| | (sulisobenzone) and the sodium salt | |
| 13 | 3-(4′-Sulfobenzylidene)bornan-2-one and salts | 58030-58-6 |
| 14 | 3-Benzylidenebornan-2-one | 16087-24-8 |
| 15 | 1-(4′-Isopropylphenyl)-3-phenylpropane-1,3-dione | 63260-25-9 |
| 16 | 4-Isopropylbenzyl salicylate | 94134-93-7 |
| 17 | 3-Imidazol-4-ylacrylic acid and its ethyl ester | 104-98-3 |
| 18 | Ethyl 2-cyano-3,3-diphenylacrylate | 5232-99-5 |
| 19 | 2′-Ethylhexyl 2-cyano-3,3-diphenylacrylate (octocrylene) | 6197-30-4 |
| 20 | Menthyl o-aminobenzoate or: | 134-09-8 |
| | 5-methyl-2-(1-methylethyl)-2-aminobenzoate | |
| 21 | Glyceryl mono-p-aminobenzoate or: | 136-44-7 |
| | 1-glyceryl 4-aminobenzoate | |
| 22 | 2,2′-Dihydroxy-4-methoxybenzophenone(dioxybenzone, | 131-53-3 |
| | benzophenone-8) | |
| 23 | 2-Hydroxy-4-methoxy-4-methylbenzophenone | 1641-17-4 |
| | (mexenone, benzophenone-10) | |
| 24 | Triethanolamine salicylate | 2174-16-5 |
| 25 | Dimethoxyphenylglyoxalic acid or: | 4732-70-1 |
| | 3,4-dimethoxyphenylglyoxalacidic sodium | |
| 26 | 3-(4′Sulfobenzylidene)bornan-2-one and its salts | 56039-58-8 |
| | (Mexoryl SL) | |
| 27 | 4-tert-Butyl-4′-methoxy-dibenzoylmethane | 70356-09-1 |
| | (BMDBM, avobenzone) | |
| 28 | 2,2′,4,4′-Tetrahydroxybenzophenone (benzophenone-2) | 131-55-5 |
| 29 | 2,2′-Methylenebis[6-(2H-benzotriazol-2-yl)-(1,1,3,3,-tetra- | 103597-45-1 |
| | methylbutyl)phenol] (bisoctyltriazole, Tinosorb M) | |
| 30 | 2,2′-(1,4-Phenylene)bis-1H-benzimidazole-4,6-disulfonic | 180898-37-7 |
| | acid, Na salt (Neo Heliopan AP) | |
| 31 | 2,4-bis[4-(2-Ethylhexyloxy)-2-hydroxy]phenyl-6-(4-methoxy- | 187393-00-6 |
| | phenyl)-(1,3,5)-triazine (Tinosorb S) | |
| 32 | 3-(4-Methylbenzylidene)camphor (MBC 95) | 36861-47-9 |
| 33 | Polyethoxylethyl 4-bis(polyethoxy)paraaminobenzoate | 113010-52-9 |
| | (PEG-25 PABA) | |
| 34 | 2,4-Dihydroxybenzophenone (benzophenone-1) | 131-56-6 |
| 35 | 2,2′-Dihydroxy-4,4′-dimethoxybenzophenone-5,5′-disodium | 3121-60-6 |
| | sulfonate (benzophenone-6) | |
| 36 | Benzoic acid, 2-[4-(diethylamino)-2-hydroxybenzoyl], hexyl | 302776-68-7 |
| | ester (Uvinul A Plus) | |
| 37 | 2-(2H-Benzotriazol-2-yl)-4-methyl-6-[2-methyl-3-[1,3,3,3-tetra- | 155633-54-8 |
| | methyl-1-[(trimethylsilyl)oxy]disiloxanyl]propyl]phenol | |
| | (silatrizole, Mexoryl XL) | |
| 38 | 1,1-[(2,2′-Dimethylpropoxy)carbonyl]-4,4-diphenyl- | 363602-15-7 |
| | 1,3-butadiene | |
| 39 | 2,4,6-Trianilino-(p-carbo-2′-ethylhexyl-1′-oxy)-1,3,5-triazine | 88122-99-0 |
| | (Uvinul T 150) | |

In a particularly preferred embodiment, the further organic UV photoprotective agent is selected from the group consisting of octocrylene (2-ethylhexyl 2-cyano-3,3-diphenyl-acrylate, CAS No. 6197-30-4), ethylhexyl salicylate (CAS No. 118-60-5), dimethicodiethyl benzalmalonate (polysilicone-15, CAS No. 207574-74-1), homosalate (CAS No. 118-56-9) and mixtures thereof.

Octocrylene is commercially available, for example, as Uvinul®N 539 T.

Ethylhexyl salicylate is commercially available, for example, as Eusolex®OS.

Dimethicodiethyl benzalmalonate is commercially available, for example, as Parsol®SLX.

Homosalate is commercially available, for example, as Eusolex®HMS.

Particular preference is accordingly given to the following combinations:
TiO₂ nanoparticles plus
derivative of dioctylbutylamidotriazone chemically bonded thereto plus
octocrylene
TiO₂ nanoparticles plus
derivative of dioctylbutylamidotriazone chemically bonded thereto plus
ethylhexyl salicylate
TiO₂ nanoparticles plus
derivative of dioctylbutylamidotriazone chemically bonded thereto plus
dimethicodiethyl benzalmalonate
TiO₂ nanoparticles plus
derivative of dioctylbutylamidotriazone chemically bonded thereto plus
homosalate
TiO₂ nanoparticles plus
derivative of tris(2-ethylhexyl) 4,4′,4″-(1,3,5-triazine-2,4,6-triyltriimino)-trisbenzoate chemically bonded thereto plus
octocrylene
TiO₂ nanoparticles plus
derivative of tris(2-ethylhexyl) 4,4′,4″-(1,3,5-triazine-2,4,6-triyltriimino)trisbenzoate chemically bonded thereto plus
ethylhexyl salicylate
TiO₂ nanoparticles plus
derivative of tris(2-ethylhexyl) 4,4′,4″-(1,3,5-triazine-2,4,6-triyltriimino)trisbenzoate chemically bonded thereto plus
dimethicodiethyl benzalmalonate
TiO₂ nanoparticles plus
derivative of tris(2-ethylhexyl) 4,4′,4″-(1,3,5-triazine-2,4,6-triyltriimino)-trisbenzoate chemically bonded thereto plus
homosalate
TiO₂ nanoparticles
derivative of hexyl 2-(4′-(diethylamino)-2′-hydroxybenzoyl)benzoate chemically bonded thereto plus
octocrylene
TiO₂ nanoparticles
derivative of hexyl 2-(4′-(diethylamino)-2′-hydroxybenzoyl)benzoate chemically bonded thereto plus
ethylhexyl salicylate
TiO₂ nanoparticles
derivative of hexyl 2-(4′-(diethylamino)-2′-hydroxybenzoyl)benzoate chemically bonded thereto plus
dimethicodiethyl benzalmalonate
TiO₂ nanoparticles
derivative of hexyl 2-(4′-(diethylamino)-2′-hydroxybenzoyl)benzoate chemically bonded thereto plus
homosalate
TiO₂ nanoparticles
4,4′-diarylbutadiene derivative chemically bonded thereto plus
octocrylene
TiO₂ nanoparticles
4,4′-diarylbutadiene derivative chemically bonded thereto plus
ethylhexyl salicylate
TiO₂ nanoparticles
4,4′-diarylbutadiene derivative chemically bonded thereto plus
dimethicodiethyl benzalmalonate
TiO₂ nanoparticles
4,4′-diarylbutadiene derivative chemically bonded thereto plus
homosalate

According to the invention, ZnO is also used as metal derivative in addition to or instead of TiO₂.

The object underlying this invention was furthermore achieved by cosmetic preparations comprising
nanoparticles of at least one metal derivative,
organic UV photoprotective agent chemically bonded thereto and
at least one further organic UV photoprotective agent which is different from that bonded to the nanoparticles and from octyl methoxycinnamate.

WO 2007/017586 and WO 2007/017587 describe how an organic UV photoprotective agent can be bonded chemically to nanoparticles of at least one metal derivative. The metal derivatives per se are likewise suitable for absorbing UV radiation. Furthermore, the metal derivatives are able to form a chemical bond with organic UV photoprotective filters.

Preferred metal derivatives are derivatives of the metals titanium, cerium, zinc, zirconium, copper and mixtures thereof, particularly preferably derivatives of titanium, cerium, copper and mixtures thereof, most preferably derivatives of titanium or zinc. Particular preference is given to derivatives of titanium.

Preferred derivatives are the oxides of the respective metals, as described in WO 2007/017587, section [0027], to which reference is hereby made in its entirety. In a particularly preferred embodiment of the invention, the abovementioned photoprotective substances are grafted chemically to the surface of titanium dioxide (TiO₂).

Analogously to section [0045] of WO 2007/017587, to which reference is hereby made in its entirety, all or some of the nanoparticles can be chemically bonded to the photoprotective substances and none or some of the nanoparticles are present without such a bond to the photoprotective substances.

The photoprotective structural element of the known UV photoprotective filters is preferably bonded to the metal derivative nanoparticles by a —COO or a —SO₂—O bond. The precursors suitable for this purpose are accessible, for example, through reaction of a metal alkoxide with the UV photoprotective filter carrying at least one —COOH or —SO₂—OH functionality (modified metal alkoxides, for example described in WO 2007/017586, sections [0059], [0062]). The UV photoprotective filter carrying the —COOH or —SO₂—OH functionality in turn is either per se commercially available or else accessible, for example, through hydrolysis of the corresponding ester.

The metal derivative nanoparticles which are chemically bonded to the photoprotective substances are then obtainable, for example, via a so-called inorganic polymerization (sol-gel process) of the suitable precursors described above. This method is described in detail in WO 2007/017586, sections [0029] to [0054], to which reference is made here in its entirety. In general, methods for producing the nanoparticles starting from metal alkoxides are described, for example, by S. Danielle, J. Mat. Chem. 13 (2003), 342-346.

The degree of functionalization, i.e. the amount of photoprotective substance based on the amount of metal derivative, can be adjusted, for example, through the ratio of metal alkoxide to modified metal alkoxide (i.e. metal alkoxide with photoprotective structural element bonded thereto) which are subjected to the sol-gel polymerization.

The table below describes photoprotective preparations according to the invention which comprise combinations of UV filters bonded to metal derivative nanoparticles and further UV filters not bonded to metal derivative nanoparticles and different from the UV filters bonded to the metal derivative nanoparticles.

| Photoprotective derivative*, | Further UV | |
|---|---|---|
| bonded to metal derivative | photoprotective | |
| (TiO₂, ZnO), of | filter | Further ingredient (optional) |
| Uvinul ®A Plus | Octocrylene | Avobenzone |
| Uvinul ®A Plus | Octocrylene | 1,1-[(2,2′-Dimethylpropoxy)carbonyl]- |
| | | 4,4-diphenyl-1,3-butadiene |
| Uvinul ®A Plus | Octocrylene | 2-(2H-Benzotriazol-2-yl)-4-methyl- |
| | | 6-[2-methyl-3-[1,3,3,3-tetramethyl- |
| | | 1-[(trimethylsilyl)oxy]disiloxanyl]propyl]- |
| | | phenol (silatrizole, Mexoryl XL) |
| Uvinul ®A Plus | Octocrylene | Homosalate |
| Uvinul ®A Plus | Octocrylene | Benzoic acid, 2-hydroxy, 2-ethylhexyl |
| | | ester |
| Uvinul ®A Plus | Octocrylene | 2-Phenylbenzimidazole-5-sulfonic |
| | | acid and its potassium, sodium and |
| | | triethanolamine salts (PBSA) |
| 1,1-[(2,2′-Dimethylpropoxy)- | Octocrylene | 2,2′-(1,4-Phenylene)bis- |
| carbonyl]-4,4-diphenyl- | | 1H-benzimidazole-4,6-disulfonic acid, |
| 1,3-butadiene | | Na salt (Neo Heliopan AP) |
| 1,1-[(2,2′-Dimethylpropoxy)- | Octocrylene | Avobenzone |
| carbonyl]-4,4-diphenyl- | | |
| 1,3-butadiene | | |
| 1,1-[(2,2′-Dimethylpropoxy)- | Octocrylene | 2,4,6-Trianilino(p-carbo-2′-ethylhexyl- |
| carbonyl]-4,4-diphenyl- | | 1′-oxy)-1,3,5-triazine |
| 1,3-butadiene | | |
| 1,1-[(2,2′-Dimethylpropoxy)- | Octocrylene | 2-(2H-Benzotriazol-2-yl)-4-methyl- |
| carbonyl]-4,4-diphenyl- | | 6-[2-methyl-3-[1,3,3,3-tetramethyl- |
| 1,3-butadiene | | 1-[(trimethylsilyl)oxy]disiloxanyl]propyl]- |
| | | phenol (silatrizole, Mexoryl XL) |
| 1,1-[(2,2′-Dimethylpropoxy)- | Octocrylene | Homosalate |
| carbonyl]-4,4-diphenyl- | | |
| 1,3-butadiene | | |
| 1,1-[(2,2′-Dimethylpropoxy)- | Octocrylene | Benzoic acid, 2-hydroxy, 2-ethylhexyl |
| carbonyl]-4,4-diphenyl- | | ester |
| 1,3-butadiene | | |
| 1,1-[(2,2′-Dimethylpropoxy)- | Octocrylene | 2-Phenylbenzimidazole-5-sulfonic |
| carbonyl]-4,4-diphenyl- | | acid and its potassium, sodium and |
| 1,3-butadiene | | triethanolamine salts (PBSA) |
| 2,4,6-Trianilino(p-carbo- | Octocrylene | 2-(2H-Benzotriazol-2-yl)-4-methyl- |
| 2′-ethylhexyl-1′-oxy)- | | 6-[2-methyl-3-[1,3,3,3-tetramethyl- |
| 1,3,5-triazine | | 1-[(trimethylsilyl)oxy]disiloxanyl]propyl] |
| | | phenol (silatrizole, Mexoryl XL) |
| 2,4,6-Trianilino(p-carbo- | Octocrylene | Avobenzone |
| 2′-ethylhexyl-1′-oxy)- | | |
| 1,3,5-triazine | | |
| 2,4,6-Trianilino(p-carbo- | Octocrylene | 2,2′-(1,4-Phenylene)bis- |
| 2′-ethylhexyl-1′-oxy)- | | 1H-benzimidazole-4,6-disulfonic acid |
| 1,3,5-triazine | | Na salt (Neo Heliopan AP) |
| 2,4,6-Trianilino(p-carbo- | Octocrylene | 2-(2H-Benzotriazol-2-yl)-4-methyl- |
| 2′-ethylhexyl-1′-oxy)- | | 6-[2-methyl-3-[1,3,3,3-tetramethyl- |
| 1,3,5-triazine | | 1-[(trimethylsilyl)oxy]disiloxanyl]propyl] |
| | | phenol (silatrizole, Mexoryl XL) |
| 2,4,6-Trianilino(p-carbo- | Octocrylene | Homosalate |
| 2′-ethylhexyl-1′-oxy)- | | |
| 1,3,5-triazine | | |
| 2,4,6-Trianilino(p-carbo- | Octocrylene | Benzoic acid, 2-hydroxy, 2-ethylhexyl |
| 2′-ethylhexyl-1′-oxy)- | | ester |
| 1,3,5-triazine | | |
| 4-Aminobenzoic acid | Octocrylene | Homosalate and/or octyl salicylate |
| 2-Phenylbenzimidazole- | Octocrylene | Homosalate and/or octyl salicylate |
| 5-sulfonic acid and its | | |
| potassium, sodium and | | |
| triethanolamine salts | | |
| 4-Methylbenzylidenecamphor | Octocrylene | Homosalate and/or octyl salicylate |
| 3-[4′Sulfobenzylidene)bornan- | Octocrylene | Homosalate and/or octyl salicylate |
| 2-one and its salts | | |
| (Mexoryl ®SL) | | |
| 2-Ethylhexyl salicylate | Octocrylene | Homosalate and/or octyl salicylate |
| 2-Cyano-3,3-diphenylacrylic | Avobenzone | Homosalate and/or octyl salicylate |
| acid, 2-ethylhexyl ester | | |
| octocrylene) | | |
| 2-Aminobenzoic acid | Octocrylene | Homosalate and/or octyl salicylate |
| carboxyaniline 1-amino- | | |
| 2-carboxybenzene | | |
| 2-Hydroxy-4-methoxy- | Octocrylene | Homosalate and/or octyl salicylate |
| benzophenone-5-sulfonic | | |
| acid | | |
| Disodium 2,2′-dihydroxy- | Octocrylene | Homosalate and/or octyl salicylate |
| 4,4′-dimethoxy-5,5′-disulfo- | | |
| benzophenone | | |
| Ethyl dihydroxypropyl PABA | Octocrylene | Homosalate and/or octyl salicylate |
| or PEG-25 PABA | and/or | |
| | avobenzone | |
| Menthyl anthranylate | Octocrylene | Homosalate and/or octyl salicylate |

*the UV photoprotective filter specified in the table is firstly converted, by hydrolysis of at least one ester bond, into an acid, via which the bonding to the nanoparticles can then take place.

Instead of TiO₂ or ZnO, other metal oxides are also suitable.

According to a preferred embodiment of the present invention, the metal derivative, in particular titanium dioxide and/or zinc oxide, which comprises at least partially photoprotective structural elements in chemically bonded form, is redispersible in a liquid medium and forms stable dispersions.

According to a preferred embodiment of the present invention, the modified nanoparticles are redispersible in polar organic solvents and form stable dispersions. This is particularly advantageous since it allows uniform incorporation, for example also in areas outside of cosmetics, such as in plastics, coatings or films.

According to a further preferred embodiment of the present invention, the modified nanoparticles are redispersible in water, where they form stable dispersions. This is particularly advantageous since this opens up the possibility of using the modified nanoparticles for example in cosmetic formulations. Also conceivable are mixtures of water and polar organic solvents.

According to a preferred embodiment of the present invention, the modified nanoparticles have a diameter of from 10 to 200 nm. This is particularly advantageous since within this size distribution, good redispersibility is ensured.

According to a further preferred embodiment of the present invention, the modified nanoparticles have a diameter of from 10 to 100 nm. This size range is particularly advantageous since, following redispersion of such modified nanoparticles, the resulting dispersions are transparent and thus, for example when added to cosmetic formulations, do not influence the coloring. Moreover, this also gives rise to the possibility of use in transparent films.

If the modified nanoparticles, in particular titanium dioxide or zinc oxide, are to be used as UV absorbers, it is advisable to use particles with a diameter of more than 5 nm since, below this limit, there is a shift in the absorption edge in the short wave range (L. Brus, J. Phys., Chem. (1986), 90, 2555-2560).

Besides one or more oil phases, the preparations for the purposes of the present invention can preferably additionally comprise one or more water phases and be present, for example, in the form of W/O, O/W, Si/W, W/Si, W/O/W or O/W/O emulsions. Such formulations may preferably also be a microemulsion (e.g. a PIT emulsion), a solids emulsion (i.e. an emulsion which is stabilized by solids, e.g. a Pickering emulsion), a sprayable emulsion or a hydrodispersion. Furthermore, for the purposes of the present invention, the preparations may also be virtually anhydrous (water content below 5% by weight based on the total weight of the preparation). The preparations according to the invention are extremely satisfactory preparations in every respect which are not restricted to a limited selection of raw materials. Accordingly, they are very particularly suitable for serving as base for preparation forms with diverse application purposes. The preparations according to the invention exhibit very good sensory and cosmetic properties, such as, for example, spreadability on the skin or the ability to absorb into the skin, and are further characterized by very good photoprotection effectiveness coupled with excellent skincare data.

The cosmetic or dermatological photoprotective preparations according to the invention can have the customary composition and serve for cosmetic or dermatological photoprotection, and also for the treatment, care and cleansing of the skin and/or of the hair and as make-up product in decorative cosmetics. In particular, the cosmetic photoprotective preparations can also be used as self-tanning preparations and then comprise substances which promote self-tanning, such as, in particular, dihydroxyacetone (CAS No. 96-26-4).

According to a further embodiment of the invention, the photoprotective preparation serves for the care or the protection of the skin, in particular for sun protection and for care in the case of exposure to sunlight and is in the form of an emulsion, a dispersion, a suspension, an aqueous surfactant preparation, a milk, a lotion, a cream, a balsam, an ointment, a gel, granules, a powder, a stick preparation, such as, for example, a lipstick, a foam, an aerosol or a spray. Such formulations are highly suitable for topical preparations. This is particularly advantageous because use in sunscreen compositions enables the UV-absorbing and the skin-calming effect for example of the zinc oxide to be utilized at the same time. Furthermore, the modified nanoparticles are very highly suited for use in sunscreen compositions since the particles can be produced in a size which appears transparent to the human eye. As a result, there is no undesired white smearing on the skin during application.

As a rule, the cosmetic composition is used for topical application to the skin. Topical preparations in this connection are to be understood as meaning those preparations which are suitable for applying the active ingredients to the skin in fine distribution and preferably in a form that can be absorbed by the skin. Of suitability for this purpose are, for example, aqueous and aqueous-alcoholic solutions, sprays, foams, foam aerosols, ointments, aqueous gels, emulsions of the O/W or W/O type, microemulsions or cosmetic stick preparations.

The UV protection performance of sunscreen compositions or of the UV filters on which they are based is generally determined in biological efficacy tests under standardized conditions. For the purposes of the present invention, “UV protection performance” means both the protection performance against UV-A radiation and also against UV-B radiation. For the purposes of the present invention, one measure of the UV protection performance is, for example, the light protection factor (LPF or SPF) or else IPD values and the like. The light protection factor (LPF, also called SPF (sun protection factor)) indicates the extension of solar irradiation which is made possible through use of the sunscreen composition. It is the quotient of erythema threshold time with sun protection composition and erythema threshold time without sun protection composition. To test the UV-A protection performance, use is usually made of the IPD method (IPD=immediate pigment darkening). Here—similar to the determination of the light protection factor, a value is determined which indicates how much longer the skin protected with the photoprotective agent can be irradiated with UV-A radiation until the pigmentation which occurs is the same as for unprotected skin.

Another test method which has become established throughout Europe is the Australian standard AS/NZS 2604:1997. In this test, the absorption of the preparation in the UV-A region is measured. In order to satisfy the standard, the preparation must absorb at least 90% of the UV-A radiation in the range from 320 to 360 nm.

According to a preferred embodiment of the invention, the photoprotective preparation comprises a cosmetically acceptable carrier. Water, an alcohol, a fat, oil or wax, a gas, a water-based liquid, a gel, an emulsion or microemulsion, a dispersion or a mixture thereof is preferred as carrier. The specified carriers exhibit good skin compatibility. Aqueous gels, emulsions or microemulsions are particularly advantageous for topical preparations.

The cosmetic and dermatological preparations according to the invention can comprise cosmetic auxiliaries, as are customarily used in such preparations, e.g. preservatives, preservation aids, bactericides, perfumes, substances for preventing foaming, dyes, pigments which have a coloring effect, thickeners, moisturizing and/or humectant substances, fillers which improve the feel on the skin, fats, oils, waxes or other customary constituents of a cosmetic or dermatological formulation, such as alcohols, polyols, polymers, foam stabilizers, electrolytes, organic solvents or silicone derivatives.

For the purposes of the present invention, advantageous preservatives are, for example, formaldehyde donors (such as, for example, DMDM hydantoin, which is available, for example, under the trade name Glydant™ (Lonza)), iodopropylbutylcarbamates (e.g. those available under the trade names Glydant®-2000, Glycacil®-L, Glycacil®-S (Lonza) and/or Dekaben®LMB (Jan Dekker)), parabens (e.g. p-hydroxybenzoic acid alkyl esters, such as methyl, ethyl, propyl and/or butyl paraben), phenoxyethanol, ethanol, benzoic acid and the like. Usually, according to the invention, the preservation system further advantageously also comprises preservation aids, such as, for example, ethylhexyloxyglycerol, glycine soya etc.

Particularly advantageous preparations are also obtained if antioxidants are used as additives or active ingredients. According to the invention, the preparations advantageously comprise one or more antioxidants. All antioxidants that are customary or suitable for cosmetic and/or dermatological applications can be used as favorable, but nevertheless optional antioxidants to be used. For the purposes of the present invention, water-soluble antioxidants can be used particularly advantageously, such as, for example, vitamins, e.g. ascorbic acid and derivatives thereof.

Preferred antioxidants are also vitamin E and derivatives thereof, and vitamin A and derivatives thereof.

The amount of antioxidants (one or more compounds) in the preparations is preferably 0.001 to 30% by weight, particularly preferably 0.05 to 20% by weight, in particular 0.1 to 10% by weight, based on the total weight of the preparation.

If vitamin E and/or derivatives thereof are the antioxidant or the antioxidants, it is advantageous to choose their respective concentrations from the range from 0.001 to 10% by weight, based on the total weight of the formulation.

If vitamin A or vitamin A derivatives, or carotenes or derivatives thereof are the antioxidant or the antioxidants, it is advantageous to choose their respective concentrations from the range from 0.001 to 10% by weight, based on the total weight of the formulation.

It is particularly advantageous if the cosmetic preparations according to the present invention comprise cosmetic or dermatological active ingredients, preferred active ingredients being antioxidants which can protect the skin from oxidative stress.

Further advantageous active ingredients for the purposes of the present invention are natural active ingredients and/or derivatives thereof, such as, for example, alpha-lipoic acid, phytoene, D-biotin, coenzyme Q10, alpha-glucosylrutin, carnitine, carnosine, natural and/or synthetic isoflavonoids, creatin, taurine and/or D-alanine.

Formulations according to the invention which comprise, for example, known antiwrinkle active ingredients, such as flavone glycosides (in particular U-glycosylrutin), coenzyme Q10, vitamin E and/or derivatives and the like are particularly advantageously suitable for the prophylaxis and treatment of cosmetic or dermatological changes in the skin as arise, for example, during skin aging (such as, for example, dryness, roughness and the formation of dryness and wrinkles, itching, reduced refitting (e.g. after washing), visible vascular dilations (teleangiectasia, cuperosis), sagging and formation of lines and wrinkles, local hyper-, hypo- and malpigmentations (e.g. age spots), increased susceptibility to mechanical stress (e.g. cracking) and the like). Furthermore, they are suitable for combating the appearance of dry and/or rough skin.

The water phase of the preparations according to the invention can advantageously comprise customary cosmetic auxiliaries, such as, for example, alcohols, in particular those of low carbon number, preferably ethanol and/or isopropanol, diols or polyols of low carbon number, and ethers thereof, preferably propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, polymers, foam stabilizers, electrolytes, dihydroxyacetone, and in particular one or more thickeners which may advantageously be selected from the group silicon dioxide, aluminum silicates, polysaccharides and derivatives thereof, e.g. hyaluronic acid, xanthan gum, hydroxypropylmethylcellulose, particularly advantageously from the group of polyacrylates, preferably a polyacrylate from the group of so-called Carbopols®, for example carbopol grades 980, 981, 1382, 2984, 5984, in each case individually or in combination. A suitable thickener from the group of polyacrylates is also Luvigel®EM (caprylic/capric triglyceride/sodium acrylate copolymer).

Also advantageous are copolymers of C₁₀₋₃₀-alkyl acrylates and one or more monomers of acrylic acid, of methacrylic acid or esters thereof.

Compounds which carry the INCI name “acrylates/C₁₀₋₃₀-alkyl “Acrylate Crosspolymer” are advantageous. Those available under the trade names Pemulen®TR1 and Pemulen®TR2 (Noveon) are particularly advantageous.

Compounds which carry the INCI name ammonium acryloyldimethyltaurate/vinylpyrrolidone copolymers are advantageous.

According to the invention, the ammonium acryloyldimethyltaurate/vinylpyrrolidone copolymers advantageously have the empirical formula [C₇H₁₆N₂SO₄]ₙ [C₆H₉NO]ₘ. Preferred species for the purposes of the present invention are listed in the Chemical Abstracts under the registry numbers (CAS No.) 58374-69-9, 13162-05-5 and 88-12-0 and are available under the trade name Aristoflex®AVC (Clariant).

Also advantageous are copolymers/crosspolymers comprising acryloyldimethyl taurates, such as, for example, Simugel®EG (Seppic).

Further thickeners to be used advantageously according to the invention are also water-soluble or -dispersible anionic polyurethanes. For the purposes of the present invention, polyurethane-1 and/or polyurethane-4, for example, are advantageous. Particularly advantageous polyurethanes for the purposes of the present invention are the grades under the trade name Avalure®UR, such as, for example, Avalure®UR 445, Avalure®UR 450 and the like. In addition, the polyurethane available under the trade name Luviset®Pur (BASF) is also advantageous for the purposes of the present invention.

Emulsifiers which can be used are nonionogenic surfactants, zwitterionic surfactants, ampholytic surfactants and anionic emulsifiers. The emulsifiers may be present in the preparation according to the invention in amounts of from 0.1 to 10% by weight, preferably 1 to 5% by weight, based on the preparation.

A surfactant from at least one of the following groups, for example, can be used as nonionic surfactant:

addition products of from 2 to 30 mol of ethylene oxide and/or 0 to 5 mol of propylene oxide onto linear fatty alcohols having 8 to 22 carbon atoms, onto fatty acids having 12 to 22 carbon atoms and onto alkylphenols having 8 to 15 carbon atoms in the alkyl group;
C_{12/18}-fatty acid mono- and diesters of addition products of from 1 to 30 mol of ethylene oxide onto glycerol;
glycerol mono- and diesters and sorbitan mono- and diesters of saturated and unsaturated fatty acids having 6 to 22 carbon atoms and ethylene oxide addition products thereof;
alkyl mono- and oligoglycosides having 8 to 22 carbon atoms in the alkyl radical and ethoxylated analogs thereof;
addition products of from 15 to 60 mol of ethylene oxide onto castor oil and/or hydrogenated castor oil;
polyol and, in particular, polyglycerol esters, such as, for example, polyglycerol polyricinoleate, polyglycerol poly-12-hydroxystearate or polyglycerol dimerate. Likewise suitable are mixtures of compounds from two or more of the substance classes;
addition products of from 2 to 15 mol of ethylene oxide onto castor oil and/or hydrogenated castor oil;
partial esters based on linear, branched, unsaturated or saturated C_{6/22}-fatty acids, ricinoleic acid and 12-hydroxystearic acid and glycerol, polyglycerol, pentaerythritol, dipentaerythritol, sugar alcohols (e.g. sorbitol), alkyl glycosides (e.g. methyl glucoside, butyl glucoside, lauryl glucoside), and polyglucosides (e.g. cellulose);
mono-, di- and trialkyl phosphates, and mono-, di- and/or tri-PEG alkyl phosphates and salts thereof;
wool wax alcohols;
polysiloxane-polyalkyl-polyether copolymers and corresponding derivatives;
mixed esters of pentaerythritol, fatty acids, citric acid and fatty alcohol as in DE PS 1165574 and/or mixed esters of fatty acids having 6 to 22 carbon atoms, methylglucose and polyols, preferably glycerol or polyglycerol and
polyalkylene glycols;
betaines.

Furthermore, zwitterionic surfactants can be used as emulsifiers. Zwitterionic surfactants is the term used to refer to those surface-active compounds which carry at least one quaternary ammonium group and at least one carboxylate or one sulfonate group in the molecule. Particularly suitable zwitterionic surfactants are the so-called betaines, such as the N-alkyl-N,N-dimethylammonium glycinates, for example cocoalkyldimethylammonium glycinate, N-acylaminopropyl-N,N-dimethylammonium glycinates, for example cocoacylaminopropyldimethylammonium glycinate, and 2-alkyl-3-carboxylmethyl-3-hydroxyethylimidazolines having in each case 8 to 18 carbon atoms in the alkyl or acyl group, and also cocoacylaminoethylhydroxyethylcarboxymethyl glycinate. Particular preference is given to the fatty acid amide derivative known under the CTFA name Cocamidopropylbetaine.

Ampholytic surfactants are likewise suitable emulsifiers. Ampholytic surfactants are understood as meaning those surface-active compounds which, apart from a C_{8,18}-alkyl or -acyl group in the molecule, comprise at least one free amino group and at least one —COOH or —SO₃H group and are capable of forming internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids having in each case about 8 to 18 carbon atoms in the alkyl group.

Particularly preferred ampholytic surfactants are N-cocoalkylaminopropionate, cocoacylaminoethylaminopropionate and C_{12/18}-acylsarcosine. Besides the ampholytic emulsifiers, quaternary emulsifiers are also suitable, particular preference being given to those of the ester quat type, preferably methyl-quaternized difatty acid triethanolamine ester salts. Furthermore, alkyl ether sulfates, monoglyceride sulfates, fatty acid sulfates, sulfosuccinates and/or ether carboxylic acids can be used as anionic emulsifiers.

Suitable oil bodies are Guerbet alcohols based on fatty alcohols having 6 to 18, preferably 8 to 10, carbon atoms, esters of linear C₆-C₂₂-fatty acids with linear C₆-C₂₂-fatty alcohols, esters of branched C₆-C₁₃-carboxylic acids with linear C₆-C₂₂-fatty alcohols, esters of linear C₆-C₂₂-fatty acids with branched alcohols, in particular 2-ethylhexanol, esters of linear and/or branched fatty acids with polyhydric alcohols (such as, for example, propylene glycol, dimerdiol or trimertriol) and/or Guerbet alcohols, triglycerides based on C₆-C₁₀-fatty acids, liquid mono-/di-, triglyceride mixtures based on C₆-C₁₈-fatty acids, esters of C₆-C₂₂-fatty alcohols and/or Guerbet alcohols with aromatic carboxylic acids, in particular benzoic acid, esters of C₂-C₁₂-dicarboxylic acids with linear or branched alcohols having 1 to 22 carbon atoms or polyols having 2 to 10 carbon atoms and 2 to 6 hydroxyl groups, vegetable oils, branched primary alcohols, substituted cyclohexanes, linear C₆-C₂₂-fatty alcohol carbonates, Guerbet carbonates, esters of benzoic acid with linear and/or branched C₆-C₂₂-alcohols (e.g. Finsolv® TN), dialkyl ethers, ring-opening products of epoxidized fatty acid esters with polyols, silicone oils and/or aliphatic or naphthenic hydrocarbons. In addition, silicone compounds can also be used as oil bodies, for example dimethylpolysiloxanes, methylphenylpolysiloxanes, cyclic silicones, and amino-, fatty-acid-, alcohol-, polyether-, epoxy-, fluorine-, alkyl- and/or glycoside-modified silicone compounds, which may be present either in liquid form or in resin form at room temperature. The oil bodies may be present in the compositions according to the invention in amounts of from 1 to 90% by weight, preferably 5 to 80% by weight, and in particular 10 to 50% by weight, based on the preparation.

In particular, reference may be made at this point in its entirety to the disclosure of WO 03/039507, p. 21, I. 25 to p. 24, I. 13.

According to a particularly preferred embodiment, the preparation according to the invention comprises further UV photoprotective filters in the form of soluble compounds or other pigments.

In an individual case, it may be desired to add further UV filter substances to the cosmetic composition or to the sun protection composition apart from the photoprotective compounds already present. This may be necessary, for example, if particular emphasis is placed on filter performance. One or more further UV photoprotective filters may be added to the preparation according to the invention.

In particular, reference may be made at this point in its entirety to the disclosure of WO 03/039507, p. 25, I. 24 to p. 29, I. 12, where further suitable UV filter substances are specified.

The UV photoprotective filters shown in the table below are suitable as further UV photoprotective filters for the preparations according to the invention.

| | | CAS No. |
|---|---|---|
| No. | Substance | (=acid) |
| 1 | 4-Aminobenzoic acid | 150-13-0 |
| 2 | 3-(4′-Trimethylammonium)benzylidenebornan-2-one methyl | 52793-97-2 |
| | sulfate | |
| 3 | 3,3,5-Trimethylcyclohexyl salicylate (homosalate) | 118-56-9 |
| 4 | 2-Hydroxy-4-methoxybenzophenone (oxybenzone) | 131-57-7 |
| 5 | 2-Phenylbenzimidazole-5-sulfonic acid and its potassium, | 27503-81-7 |
| | sodium and triethanolamine salts | |
| 6 | 3,3′-(1,4-Phenylenedimethine)bis(7,7-dimethyl-2-oxo- | 90457-82-2 |
| | bicyclo[2.2.1]heptane-1-methanesulfonic acid) and its salts | |
| 7 | Polyethoxyethyl 4-bis(polyethoxy)aminobenzoate | 113010-52-9 |
| 8 | 2-Ethylhexyl 4-dimethylaminobenzoate | 21245-02-3 |
| 9 | 2-Ethylhexyl salicylate | 118-60-5 |
| 10 | 2-Isoamyl 4-methoxycinnamate | 71617-10-2 |
| 12 | 2-Hydroxy-4-methoxybenzophenone-5-sulfonic acid | 4065-45-6 |
| | (sulisobenzone) and the sodium salt | |
| 13 | 3-(4′-Sulfobenzylidene)bornan-2-one and salts | 58030-58-6 |
| 14 | 3-Benzylidenebornan-2-one | 16087-24-8 |
| 15 | 1-(4′-Isopropylphenyl)-3-phenylpropane-1,3-dione | 63260-25-9 |
| 16 | 4-Isopropylbenzyl salicylate | 94134-93-7 |
| 17 | 3-Imidazol-4-ylacrylic acid and its ethyl ester | 104-98-3 |
| 18 | Ethyl 2-cyano-3,3-diphenylacrylate | 5232-99-5 |
| 19 | 2′-Ethylhexyl 2-cyano-3,3-diphenylacrylate (octocrylene) | 6197-30-4 |
| 20 | Menthyl o-aminobenzoate or: | 134-09-8 |
| | 5-methyl-2-(1-methylethyl)-2-aminobenzoate | |
| 21 | Glyceryl mono-p-aminobenzoate or: | 136-44-7 |
| | 1-glyceryl 4-aminobenzoate | |
| 22 | 2,2′-Dihydroxy-4-methoxybenzophenone (dioxybenzone, | 131-53-3 |
| | benzophenone-8) | |
| 23 | 2-Hydroxy-4-methoxy-4-methylbenzophenone | 1641-17-4 |
| | (mexenone, benzophenone-10) | |
| 24 | Triethanolamine salicylate | 2174-16-5 |
| 25 | Dimethoxyphenylglyoxalic acid or: | 4732-70-1 |
| | 3,4-dimethoxyphenylglyoxalacidic sodium | |
| 26 | 3-(4′Sulfobenzylidene)bornan-2-one and its salts | 56039-58-8 |
| | (Mexoryl SL) | |
| 27 | 4-tert-Butyl-4′-methoxydibenzoylmethane | 70356-09-1 |
| | (BMDBM, avobenzone) | |
| 28 | 2,2′,4,4′-Tetrahydroxybenzophenone (benzophenone-2) | 131-55-5 |
| 29 | 2,2′-Methylenebis[6-(2H-benzotriazol-2-yl)-4-(1,1,3,3-tetra- | 103597-45-1 |
| | methylbutyl)phenol] (bisoctyltriazole, Tinosorb M) | |
| 30 | 2,2′-(1,4-Phenylene)bis-1H-benzimidazole-4,6-disulfonic | 180898-37-7 |
| | acid, Na salt (Neo Heliopan AP) | |
| 31 | 2,4-bis[4-(2-Ethylhexyloxy)-2-hydroxy]phenyl-6-(4-methoxy- | 187393-00-6 |
| | phenyl)-(1,3,5)-triazine (Tinosorb S) | |
| 32 | 3-(4-Methylbenzylidene)camphor (MBC 95) | 36861-47-9 |
| 33 | Polyethoxylethyl 4-bis(polyethoxy)paraaminobenzoate | 113010-52-9 |
| | (PEG-25 PABA) | |
| 34 | 2,4-Dihydroxybenzophenone (benzophenone-1) | 131-56-6 |
| 35 | 2,2′-Dihydroxy-4,4′-dimethoxybenzophenone-5,5′-disodium | 3121-60-6 |
| | sulfonate (benzophenone-6) | |
| 36 | Benzoic acid, 2-[4-(diethylamino)-2-hydroxybenzoyl], hexyl | 302776-68-7 |
| | ester (Uvinul A Plus) | |
| 37 | 2-(2H-Benzotriazol-2-yl)-4-methyl-6-[2-methyl-3-[1,3,3,3-tetra- | 155633-54-8 |
| | methyl-1-[(trimethylsilyl)oxy]disiloxanyl]propyl]phenol | |
| | (silatrizole, Mexoryl XL) | |
| 38 | 1,1-[(2,2′-Dimethylpropoxy)carbonyl]-4,4-diphenyl- | 363602-15-7 |
| | 1,3-butadiene | |
| 39 | 2,4,6-Trianilino-(p-carbo-2′-ethylhexy]-1′-oxy)-1,3,5-triazine | 88122-99-0 |
| | (Uvinul T 150) | |

Oil-soluble UV-B filters which can be used are, for example, the following substances:

3-benzylidenecamphor and derivatives thereof, e.g. 3-(4-methylbenzylidene)camphor; 4-aminobenzoic acid derivatives, preferably 2-ethylhexyl 4-(dimethylamino)benzoate, 2-octyl 4-(dimethylamino)benzoate and amyl 4-(dimethylamino)benzoate;
esters of cinnamic acid, preferably 2-ethylhexyl 4-methoxycinnamate, propyl 4-methoxycinnamate, isoamyl 4-methoxycinnamate, isopentyl 4-methoxycinnamate, 2-ethylhexyl 2-cyano-3-phenylcinnamate (octocrylene);
esters of salicylic acid, preferably 2-ethylhexyl salicylate, 4-isopropylbenzyl salicylate, homomethyl salicylate;
derivatives of benzophenone, preferably 2-hydroxy-4-methoxybenzophenone, 2-hydroxy-4-methoxy-4′-methylbenzophenone, 2,2′-dihydroxy-4-methoxybenzophenone;
esters of benzylmalonic acid, preferably di-2-ethylhexyl 4-Methoxybenzomalonate;
triazine derivatives, such as, for example, 2,4,6-trianilino-(p-carbo-2′-ethyl-1′-hexyloxy)-1,3,5-triazine (octyltriazone) and dioctylbutamidotriazone (Uvasorb® HEB);
propane-1,3-diones, such as, for example, 1-(4-tert-butylphenyl)-3-(4′-methoxyphenyl)propane-1,3-dione;
2-phenylbenzimidazole-5-sulfonic acid and alkali metal, alkaline earth metal, ammonium, alkylammonium, alkanolammonium and glucoammonium salts thereof;
sulfonic acid derivatives of benzophenones, preferably 2-hydroxy-4-methoxybenzophenone-5-sulfonic acid and its salts;
sulfonic acid derivatives of 3-benzylidenecamphor, such as, for example, 4-(2-oxo-3-bornylidenemethyl)benzenesulfonic acid and 2-methyl-5-(2-oxo-3-bornylidene)sulfonic acid and salts thereof.

Particular preference is given to the use of esters of cinnamic acid, preferably 2-ethylhexyl 4-methoxycinnamate, isopentyl 4-methoxycinnamate, 2-ethylhexyl 2-cyano-3-phenylcinnamate (octocrylene).

Furthermore, the use of derivatives of benzophenone, in particular 2-hydroxy-4-methoxybenzophenone, 2-Hydroxy-4-methoxy-4′-methylbenzophenone, 2,2′-dihydroxy-4-methoxybenzophenone and the use of propane-1,3-diones, such as, for example, 1-(4-tert-butylphenyl)-3-(4′-methoxyphenyl)propane-1,3-dione is preferred.

Suitable typical UV-A filters are:

derivatives of benzoylmethane, such as, for example, 1-(4′-tert-butylphenyl)-3-(4′-methoxyphenyl)propane-1,3-dione, 4-tert-butyl-4′-methoxydibenzoylmethane or 1-phenyl-3-(4′-isopropylphenyl)propane-1,3-dione;
aminohydroxy-substituted derivatives of benzophenones, such as, for example, N,N-diethylaminohydroxybenzoyl n-hexylbenzoate;
2,2′-(1,4-phenylene)bis-1H-benzimidazole-4,6-disulfonic acid, Na salt (Neo Heliopan®AP, CAS No. 180898-37-7).

The UV-A and UV-B filters can of course also be used in mixtures.

Further photoprotective filters which can be used are, however, also other insoluble pigments, e.g. finely disperse metal oxides or salts, such as, for example, titanium dioxide, iron oxide, aluminum oxide, cerium oxide, zirconium oxide, silicates (talc), barium sulfate and zinc stearate. The particles here should have an average diameter of less than 200 nm, preferably less than 100 nm.

Besides the two abovementioned groups of primary photoprotective substances, it is also possible to use secondary photoprotective agents of the antioxidant type which interrupt the photochemical reaction chain which is triggered when UV radiation penetrates into the skin. Typical examples thereof are superoxide dismutase, tocopherols (vitamin E) and ascorbic acid (vitamin C).

The total content of the photoprotective agents in the sun protection composition according to the invention is usually 1 to 20% by weight, preferably 5 to 15% by weight.

A preferred preparation according to the invention as such can comprise 1 to 95% by weight, preferably 5 to 80% by weight, and in particular 10 to 60% by weight, of water.

According to a particularly preferred embodiment, the cosmetic preparation according to the invention further comprises care substances, further cosmetic active ingredients and/or auxiliaries and additives.

The further cosmetic active ingredients used are, in particular, skin moisturizers, antimicrobial substances and/or deodorizing or antiperspirant substances. This has the advantage that further desired effects can be achieved which contribute to the care or treatment of the skin or, for example, increase the wellbeing of the user of the cosmetic preparation when using this preparation.

Thus, besides the carrier, the metal derivative nanoparticles, and the photoprotective compounds bonded with them, water and physiologically suitable solvents, care constituents, such as, for example, oils, waxes, fats, refatting substances, thickeners, emulsifiers and fragrances, inter alia, may also be present in the cosmetic preparation. A high content of care substances is advantageous in particular for the topical prophylactic or cosmetic treatment of the skin.

It is particularly advantageous if, besides the animal and vegetable fats and oils, that in many cases likewise have a care effect, the preparation also comprises further care components. The group of care active ingredients which can be used comprises, for example: fatty alcohols having 8-22 carbon atoms, in particular fatty alcohols of natural fatty acids; animal and vegetable protein hydrolyzates, in particular elastin, collagen, keratin, milk protein, soya protein, silk protein, oat protein, pea protein, almond protein and wheat protein hydrolysates; vitamins and vitamin precursors, in particular those of vitamin groups A and B; mono-, di- and oligosaccharides; plant extracts; honey extracts; ceramides; phospholipids; vaseline, paraffin and silicone oils; fatty acid and fatty alcohol esters, in particular the monoesters of fatty acids with alcohols having 3-24 carbon atoms. The vitamins, provitamins or vitamin precursors to be used with preference in the preparation according to the invention include, inter alia:

vitamins, provitamins and vitamin precursors from groups A, C, E and F, in particular 3,4-didehydroretinol, β-carotene (provitamin of vitamin A), ascorbic acid (vitamin C), and palmitic acid esters, glucosides or phosphates of ascorbic acid, tocopherols, in particular α-tocopherol and its esters, e.g. the acetate, the nicotinate, the phosphate and the succinate; also vitamin F, which is understood as meaning essential fatty acids, particularly linoleic acid, linolenic acid and arachidonic acid;
vitamin A and its derivatives and provitamins advantageously exhibit a particularly skin-smoothing effect.

The vitamins, provitamins or vitamin precursors of the vitamin B group or derivatives thereof and also the derivatives of 2-furanone to be used with preference in the preparation according to the invention include, inter alia:

vitamin B₁, trivial name thiamine, chemical name 3-[(4′-amino-2′-methyl-5′-pyrimidinyl)-methyl]-5-(2-hydroxyethyl)-4-methylthiazolium chloride. Preference is given to using thiamine hydrochloride in amounts of from 0.05 to 1% by weight, based on the total composition.

Vitamin B₂, trivial name riboflavin, chemical name 7,8-dimethyl-10-(1-D-ribityl)benzo[g]pteridine-2,4(3H,10H)-dione. In free form, riboflavin occurs, for example, in whey, other riboflavin derivatives can be isolated from bacteria and yeasts. A stereoisomer of riboflavin likewise suitable according to the invention is lyxoflavin, which can be isolated from fishmeal or liver and carries a D-arabityl radical instead of the D-ribityl. Preference is given to using riboflavin or its derivatives in amounts of from 0.05 to 1% by weight, based on the total composition.

Vitamin B₃. The compounds nicotinic acid and nicotinamide (niacinamide) often bear this name. According to the invention, preference is given to nicotinamide, which is present in the compositions according to the invention preferably in amounts of from 0.05 to 1% by weight, based on the total composition.

Vitamin B₅ (pantothenic acid and panthenol). Preference is given to using panthenol. Derivatives of panthenol that can be used according to the invention are, in particular, the esters and ethers of panthenol, and also cationically derivatized panthenols. In a further preferred embodiment of the invention, it is also possible to use derivatives of 2-furanone in addition to pantothenic acid or panthenol. Particularly preferred derivatives are the also commercially available substances dihydro-3 hydroxy-4,4-dimethyl-2(3H)-furanone with the trivial name pantolactone (Merck), 4 hydroxymethyl-γ-butyrolactone (Merck), 3,3-dimethyl-2-hydroxy-γ-butyrolactone (Aldrich) and 2,5-dihydro-5-methoxy-2-furanone (Merck), with all stereoisomers being expressly included.

These compounds advantageously impart moisturizing and skin-calming properties to the cosmetic preparation according to the invention.

The specified compounds of the vitamin B₅ type and the 2-furanone derivatives are present in the compositions according to the invention preferably in a total amount of from 0.05 to 10% by weight, based on the total composition. Total amounts of from 0.1 to 5% by weight are particularly preferred.

Vitamin B₆, which is understood here as not meaning a single substance, but the derivatives of 5-hydroxymethyl-2-methylpyridin-3-ol known under the trivial names pyridoxin, pyridoxamine and pyridoxal. Vitamin B₆ is present in the compositions according to the invention preferably in amounts of from 0.0001 to 1.0% by weight, in particular in amounts of from 0.001 to 0.01% by weight.

Vitamin B₇ (biotin), also referred to as vitamin H or “skin vitamin”. Biotin is (3aS,4S,6aR)-2-oxohexahydrothienol[3,4-d]imidazole-4-valeric acid. Biotin is present in the compositions according to the invention preferably in amounts of from 0.0001 to 1.0% by weight, in particular in amounts of from 0.001 to 0.01% by weight.

According to the invention, panthenol, pantolactone, nicotinamide and biotin are very particularly preferred.

Auxiliaries and additives are to be understood as meaning substances which are suitable for improving the esthetic, application and/or cosmetic properties, such as, for example, coemulsifiers, organic solvents, superfatting agents, stabilizers, antioxidants, waxes or fats, consistency regulators, thickeners, tanning agents, vitamins, cationic polymers, biogenic active ingredients, preservatives, hydrotropes, solubilizers, dyes and fragrances.

For example, the following auxiliaries and additives can be used:
Allantoin,
AloeVera,
Bisabolol,
Ceramides and psuedoceramides.

Antioxidants advantageously improve the stability of the preparations according to the invention. Antioxidants are, for example, amino acids (e.g. glycine, histidine, tyrosine, tryptophan) and derivatives thereof, imidazole and imidazole derivatives (e.g. urocanic acid), peptides, such as, for example, D,L-carnosine, D-carnosine, L-carnosine and derivatives thereof (e.g. anserine), carotenoids, carotenes (e.g. α-carotene, (β-carotene, lycopene) and derivatives thereof, lipoic acid and derivatives thereof (e.g. dihydrolipoic acid), aurothioglucose, propylthiouracil and other thio compounds (e.g. thioglycerol, thiosorbitol, thioglycolic acid, thioredoxin, glutathione, cystein, cystine, cystamine and the glycosyl-, N-acetyl, methyl, ethyl, propyl, amyl, butyl, lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocystein sulfoximine, buthionine sulfones, penta-, hexa-, heptathionine sulfoximine) in very low tolerated doses (e.g. pmol/kg to pmol/kg), also metal chelators (e.g. α-hydroxy fatty acids, EDTA, EGTA, phytic acid, lactoferrin), α-hydroxy acids (e.g. citric acid, lactic acid, malic acid), humic acids, bile acid, bile extracts, gallic acid esters (e.g. propyl, octyl and dodecyl gallate), flavonoids, catechins, bilirubin, biliverdin and derivatives thereof, unsaturated fatty acids and derivatives thereof (e.g. γ-linolenic acid, linoleic acid, arachidonic acid, oleic acid), folic acid and derivatives thereof, hydroquinone and derivatives thereof (e.g. arbutin), ubiquinone and ubiquinol, and derivatives thereof, vitamin C and derivatives thereof (e.g. ascorbyl palmitate, stearate, dipalmitate, acetate, Mg-ascorbyl phosphates, sodium and magnesium ascorbate, disodium ascorbyl phosphate and sulfate, potassium ascorbyl tocopheryl phosphate, chitosan ascorbate), isoascorbic acid and derivatives thereof, tocopherols and derivatives thereof (e.g. tocopheryl acetate, linoleate, oleate and succinate, tocophereth-5, tocophereth-10, tocophereth-12, tocophereth-18, tocophereth-50, tocophersolan), vitamin A and derivatives (e.g. vitamin A palmitate), the coniferyl benzoate of benzoic resin, rutin, rutinic acid and derivatives thereof, disodium rutinyl disulfate, cinnamic acid and derivatives thereof (e.g. ferulic acid, ethyl ferulate, caffeic acid), kojic acid, chitosan glycolate and salicylate, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and zinc derivatives (e.g. ZnO, ZnSO₄), selenium and selenium derivatives (e.g. selenomethionine), stilbenes and stilbene derivatives (e.g. stilbene oxide, trans-stilbene oxide).

According to the invention, suitable derivatives (salts, esters, sugars, nucleotides, nucleosides, peptides and lipids) and mixtures of these specified active ingredients or plant extracts (e.g. tea tree oil, rosemary extract and rosemarinic acid), which comprise these antioxidants can be used. Preferred lipophilic, oil-soluble antioxidants from this group are tocopherol and derivatives thereof, gallic acid esters, flavonoids and carotenoids, and butylhydroxytoluene/anisole. Preferred water-soluble antioxidants are amino acids, e.g. tyrosine and cystein and derivatives thereof, and also tannins, in particular those of vegetable origin. The total amount of the antioxidants in the cosmetic preparations according to the invention is 0.001-20% by weight, preferably 0.05-10% by weight, in particular 0.1-5% by weight and very particularly preferably 0.1 to 2% by weight.

Triterpenes, in particular triterpenoic acids such as ursolic acid, rosemarinic acid, betulinic acid, boswellic acid and byronolic acid,

monomeric catechins, particularly catechin and epicatechin, leukoanthocyanidins, catechin polymers (catechin tannins) and gallotannins,
thickeners, e.g. gelatin, plant gums such as agar agar, guar gum, alginates, xanthan gum, gum arable, karaya gum or carob seed flour, natural and synthetic clays and sheet silicates, e.g. bentonite, hectorite, montmorillonite or Laponite®, completely synthetic hydrocolloids, such as, for example, polyvinyl alcohol, and furthermore Ca, Mg or Zn soaps of fatty acids,
plant glycosides,
structurants, such as maleic acid and lactic acid,
dimethyl isosorbide,
alpha-, beta- and gamma-cyclodextrins, in particular for stabilizing retinol,
solvents, swelling and penetration substances, such as ethanol, isopropanol, ethylene glycol, propylene glycol, propylene glycol monoethyl ether, glycerol and diethylene glycol, carbonates, hydrogen carbonates, guanidines, ureas, and primary, secondary and tertiary phosphates
perfume oils, pigments and dyes for coloring the composition,
substances for adjusting the pH, e.g. α- and β-hydroxycarboxylic acids, complexing agents, such as EDTA, NTA, β-alaninediacetic acid and phosphoric acids, opacifiers, such as latex, styrene/PVP and styrene/acrylamide copolymers, pearlizing agents, such as ethylene glycol mono- and distearate, and PEG-3 distearate,
propellants, such as propane/butane mixtures, N₂O, dimethyl ether, CO₂ and air.

The addition of allantoin, bisabolol and/or aloe vera, including in the form of extracts, to the cosmetic preparations according to the invention further improves the skin-calming, moisturizing and skincare properties of the formulations and is therefore particularly preferred.

As further ingredients, the cosmetic preparation according to the invention can comprise further surfactants compatible with the other ingredients in secondary amounts.

Typical examples of anionic surfactants are soaps, alkylbenzenesulfonates, alkanesulfonates, olefinsulfonates, alkyl ether sulfonates, glycerol ether sulfonates, α-methyl ester sulfonates, sulfo fatty acids, alkyl sulfates, fatty alcohol ether sulfates, glycerol ether sulfates, hydroxy mixed ether sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids, such as, for example, acyl lactylates, acyl tartrates, acyl glutamates and acyl aspartates, alkyl oligoglucoside sulfates, protein fatty acid condensates (in particular vegetable products based on wheat) and alkyl (ether) phosphates.

If the anionic surfactants comprise polyglycol ether chains, these can have a conventional homolog distribution, but preferably have a narrowed homolog distribution.

Typical examples of nonionic surfactants are fatty alcohol polyglycol ethers, fatty acid polyglycol esters, fatty acid amide polyglycol ethers, fatty amine polyglycol ethers, alkoxylated triglycerides, mixed ethers or mixed formals, if appropriate partially oxidized alk(en)yl oligoglycosides and glucoronic acid derivatives, fatty acid N-alkylglucamides, protein hydrolyzates (in particular vegetable products based on wheat), polyol fatty acid esters, sugar esters, sorbitan esters, polysorbates and amine oxides. If the nonionic surfactants comprise polyglycol ether chains, these can have a conventional homolog distribution, but preferably have a narrowed homolog distribution.

Typical examples of cationic surfactants are quaternary ammonium compounds and ester quats, in particular quaternized fatty acid trialkanolamine ester salts.

Typical examples of amphoteric and zwitterionic surfactants are alkylbetaines, alkylamidobetaines, aminopropionates, aminoglycinates, imidazolinium betaines and sulfobetaines.

The use of the photoprotective preparations according to the invention is in particular likewise possible in hair cosmetics such as shampoos, conditioners, rinses, hair tonics, hair gel, hair spray etc. In particular leave-on products, which remain on the hair and/or the scalp following application are particularly highly suitable. The preparation thus applied to the scalp and the hair can thus also act there as UV protectant and/or develop its skin-calming effect on the scalp.

The preparations according to the invention can also be used in the field of washing, showering and bath preparations.

According to a preferred embodiment of the cosmetic composition according to the invention, the cosmetic composition is applied to the surface of the body to be treated and/or to be protected, i.e. topically. This application form is particularly advantageous since it is easy to handle, meaning that incorrect dosages are largely excluded. Furthermore, an additional care effect for the skin can be achieved. If only individual parts of the body are exposed to solar radiation, the sunscreen composition can, moreover, be applied only specifically to these parts of the body.

The present invention further provides the use of the metal oxides surface-modified according to the invention for UV protection. This is particularly advantageous since, on account of the finely divided nature of, for example, the surface-modified metal oxide and the good distribution, a particularly high UV absorption is achieved.

The present invention further provides the use of the metal oxides surface-modified according to the invention, in particular of zinc oxide, as antimicrobial active ingredient. The use of these particles is particularly advantageous for this use purpose since, on account of the finely divided nature of the particles and the resulting large surface area, the antimicrobial effect is greatly improved and, on the other hand, on account of the good dispersion properties of the material, the zinc oxide is present in finely divided form. The zinc oxide can therefore be used without problems in various supply forms, such as, for example, creams, skin milk, lotions or tonics.

The present invention further provides a pharmaceutical composition which comprises a surface-modified metal oxide according to the invention. This pharmaceutical composition is characterized by the fact that, on account of the finely divided nature of the particles, the pharmaceutical effectiveness is greatly increased.

Moreover, the pharmaceutical composition according to the invention has the advantage that, on account of the good long-term stability, already described above, of, for example, the zinc oxide dispersions, it is possible to dispense with the addition of stabilizers which prevent separation. The compatibility of the pharmaceutical composition is thus additionally increased.

The invention will be illustrated in more detail by reference to the following examples.

### EXAMPLES OF COSMETIC FORMULATIONS

The following preparations according to the invention comprise, as constituent, modified metal derivative nanoparticles.

In the examples, specified below, of cosmetic preparations according to the invention, the term “hybrid” is at least one combination selected from:

1)—Nanoparticles of at least one metal derivative
at least one triazine derivative chemically bonded to the nanoparticles of at least one metal derivative (TiO₂ or ZnO).
   2)—Nanoparticles of at least one metal derivative
at least one hydroxybenzophenone derivative chemically bonded to the nanoparticles of at least one metal derivative (TiO₂ or ZnO).
   3)—Nanoparticles of at least one metal derivative
at least one compound chemically bonded to the nanoparticles of at least one metal derivative (TiO₂ or ZnO) and selected from the photoprotective compounds specified in WO 2007/017587, sections [0036] and [0037].
   4)—Nanoparticles of at least one metal derivative
at least one 4,4′-diarylbutadiene derivative chemically bonded to the nanoparticles of at least one metal derivative (TiO₂ or ZnO).

The quantitative data below are in parts by weight. The way in which the preparations can be prepared is known to the person skilled in the art.

Preparation 1

| | | |
|---|---|---|
| A | 7.50 Uvinul ®MC 80 | Ethylhexyl Cinnamate |
| | 2.00 Uvinul ®M 40 | Benzophenone-3 |
| | 0.80 Rylo ®PG 11 | Polyglyceryl Dimer Soyate |
| | 1.00 Span ®60 | Sorbitan Stearate |
| | 0.50 Vitamin E acetate | Tocopheryl Acetate |
| | 3.00 Dracorin ®100 SE | Glyceryl Stearate, PEG-100 Stearate |
| | 1.00 Cremophor ®CO 410 | PEG-40 Hydrogenated Castor Oil |
| B | 3.00 Hybrid | |
| | 1.00 Cetiol ®SB 45 | Butyrospermum Parkii (Shea Butter) |
| | 6.50 Finsolv ®TN | C12-15 Alkyl Benzoate |
| C | 5.00 Butylene glycol | Butylene Glycol |
| | 0.30 Keltrol ® | Xanthan Gum |
| | 0.10 Edeta BD | Disodium EDTA |
| | 0.10 Allantoin | Allantoin |
| | 67.20 Water dem. | Aqua dem. |
| D | 1.00 Sepigel ®305 | Polyacrylamide, C13-14 |
| | | Isoparaffin, Laureth-7 |
| | q.s. Preservative | |

Preparation 2

| | | |
|---|---|---|
| A | 7.50 Uvinul MC 80 | Ethylhexyl Cinnamate |
| | 2.00 Uvinul M 40 | Benzophenone-3 |
| | 0.80 Rylo PG 11 | Polyglyceryl Dimer Soyate |
| | 1.00 Span 60 | Sorbitan Stearate |
| | 0.50 Vitamin E acetate | Tocopheryl Acetate |
| | 3.00 Dracorin 100 SE | Glyceryl Stearate, PEG-100 Stearate |
| | 1.00 Cremophor CO 410 | PEG-40 Hydrogenated Castor Oil |
| B | 3.00 Hybrid | |
| | 1.00 Cetiol SB 45 | Butyrospermum Parkii (Shea Butter) |
| | 6.50 Finsolv TN | C12-15 Alkyl Benzoate |
| C | 5.00 Butylene glycol | Butylene Glycol |
| | 0.30 Keltrol | Xanthan Gum |
| | 0.10 Edeta BD | Disodium EDTA |
| | 0.10 Allantoin | Allantoin |
| | 67.20 Water dem. | Aqua dem. |
| D | 2.00 Luvigel ®EM | Caprylic/Capric Triglyceride and |
| | | Sodium Acrylates Copolymer |

Preparation 3

| | | |
|---|---|---|
| A | 7.50 Uvinul MC 80 | Ethylhexyl Cinnamate |
| | 2.00 Uvinul A Plus | Diethylamino Hydroxynezoyl |
| | | Hexyl Benzoate |
| | 0.80 Rylo PG 11 | Polyglyceryl Dimer Soyate |
| | 1.00 Span 60 | Sorbitan Stearate |
| | 0.50 Vitamin E acetate | Tocopheryl Acetate |
| | 3.00 Dracorin 100 SE | Glyceryl Stearate, PEG-100 Stearate |
| | 1.00 Cremophor CO 410 | PEG-40 Hydrogenated Castor Oil |
| B | 3.00 Hybrid | |
| | 1.00 Cetiol SB 45 | Butyrospermum Parkii (Shea Butter) |
| | 6.50 Finsolv TN | C12-15 Alkyl Benzoate |
| C | 5.00 Butylene glycol | Butylene Glycol |
| | 0.30 Keltrol | Xanthan Gum |
| | 0.10 Edeta BD | Disodium EDTA |
| | 0.10 Allantoin | Allantoin |
| | 67.20 Water dem. | Aqua dem. |
| D | 2.00 Luvigel EM | Caprylic/Capric Triglyceride and |
| | | Sodium Acrylates Copolymer |
| | q.s. Preservative | |

Preparation 4

| | | |
|---|---|---|
| A | 4.00 Uvinul MC 80 | Ethylhexyl Cinnamate |
| | 2.00 Uvinul ®T 150 | Ethyl Hexyl Triazone |
| | 2.00 Uvinul ®A Plus | Diethylamino Hydroxybenzoyl |
| | | Hexyl Benzoate |
| | 0.80 Rylo PG 11 | Polyglyceryl Dimer Soyate |
| | 1.00 Span ®60 | Sorbitan Stearate |
| | 0.50 Vitamin E acetate | Tocopheryl Acetate |
| | 3.00 Dracorin 100 SE | Glyceryl Stearate, PEG-100 Stearate |
| | 1.00 Cremophor CO 410 | PEG-40 Hydrogenated Castor Oil |
| B | 3.00 Hybrid | |
| | 1.00 Cetiol SB 45 | Butyrospermum Parkii (Shea Butter) |
| | 6.50 Finsolv TN | C12-15 Alkyl Benzoate |
| C | 5.00 Butylene glycol | Butylene Glycol |
| | 0.30 Keltrol | Xanthan Gum |
| | 0.10 Edeta BD | Disodium EDTA |
| | 0.10 Allantoin | Allantoin |
| | 67.20 Water dem. | Aqua dem. |
| D | 2.00 Luvigel EM | Caprylic/Capric Triglyceride and |
| | | Sodium Acrylates Copolymer |
| | q.s. Preservative | |

Preparation 5

| | | |
|---|---|---|
| A | 4.00 Uvinul MC 80 | Ethylhexyl Cinnamate |
| | 2.00 Uvinul T 150 | Ethyl Hexyl Triazone |
| | 2.00 Uvinul A Plus | Diethylamino Hydroxybenzoyl |
| | | Hexyl Benzoate |
| | 0.80 Rylo PG 11 | Polyglyceryl Dimer Soyate |
| | 1.00 Span 60 | Sorbitan Stearate |
| | 0.50 Vitamin E acetate | Tocopheryl Acetate |
| | 3.00 Dracorin 100 SE | Glyceryl Stearate, PEG-100 Stearate |
| | 1.00 Cremophor CO 410 | PEG-40 Hydrogenated Castor Oil |
| B | 3.00 Hybrid | |
| | 1.00 Cetiol SB 45 | Butyrospermum Parkii (Shea Butter) |
| | 6.50 Finsolv TN | C12-15 Alkyl Benzoate |
| C | 5.00 Butylene glycol | Butylene Glycol |
| | 0.30 Keltrol | Xanthan Gum |
| | 0.10 Edeta BD | Disodium EDTA |
| | 0.10 Allantoin | Allantoin |
| | 67.20 Water dem. | Aqua dem. |
| D | 2.00 Luvigel EM | Caprylic/Capric Triglyceride and |
| | | Sodium Acrylates Copolymer |
| | q.s. Preservative | |

Preparation 6

| | | |
|---|---|---|
| A | 4.00 Uvinul MC 80 | Ethylhexyl Cinnamate |
| | 2.00 Uvinul T 150 | Ethyl Hexyl Triazone |
| | 2.00 Uvinul A Plus | Diethylamino Hydroxynezoyl |
| | | Hexyl Benzoate |
| | 0.80 Rylo PG 11 | Polyglyceryl Dimer Soyate |
| | 1.00 Span 60 | Sorbitan Stearate |
| | 0.50 Vitamin E acetate | Tocopheryl Acetate |
| | 3.00 Dracorin 100 SE | Glyceryl Stearate, PEG-100 Stearate |
| | 1.00 Cremophor CO 410 | PEG-40 Hydrogenated Castor Oil |
| B | 3.00 Hybrid | |
| | 1.00 Cetiol SB 45 | Butyrospermum Parkii (Shea Butter) |
| | 6.50 Finsolv TN | C12-15 Alkyl Benzoate |
| C | 5.00 Butylene glycol | Butylene Glycol |
| | 0.30 Keltrol | Xanthan Gum |
| | 0.10 Edeta BD | Disodium EDTA |
| | 0.10 Allantoin | Allantoin |
| | 67.20 Water dem. | Aqua dem. |
| D | 2.00 Luvigel EM | Caprylic/Capric Triglyceride and |
| | | Sodium Acrylates Copolymer |

Preparation 7

| | | |
|---|---|---|
| A | 4.00 Uvinul MC 80 | Ethylhexyl Cinnamate |
| | 1.00 Uvinul T 150 | Ethyl Hexyl Triazone |
| | 2.00 Parsol 1789 | Butyl Methoxydibenzoylmethane |
| | 0.80 Rylo PG 11 | Polyglyceryl Dimer Soyate |
| | 1.00 Span 60 | Sorbitan Stearate |
| | 0.50 Vitamin E acetate | Tocopheryl Acetate |
| | 3.00 Dracorin 100 SE | Glyceryl Stearate, PEG-100 Stearate |
| | 1.00 Cremophor CO 410 | PEG-40 Hydrogenated Castor Oil |
| B | 3.00 Hybrid | |
| | 1.00 Cetiol SB 45 | Butyrospermum Parkii (Shea Butter) |
| | 6.50 Finsolv TN | C12-15 Alkyl Benzoate |
| C | 5.00 Butylene glycol | Butylene Glycol |
| | 0.30 Keltrol | Xanthan Gum |
| | 0.10 Edeta BD | Disodium EDTA |
| | 0.10 Allantoin | Allantoin |
| | 67.20 Water dem. | Aqua dem. |
| D | 2.00 Luvigel EM | Caprylic/Capric Triglyceride and |
| | | Sodium Acrylates Copolymer |
| | q.s. Preservative | |

Preparation 8

| | | |
|---|---|---|
| A | 4.00 Uvinul MC 80 | Ethylhexyl Cinnamate |
| | 1.00 Uvinui T 150 | Ethyl Hexyl Triazone |
| | 2.00 Parsol 1789 | Butyl Methoxydibenzoylmethane |
| | 0.80 Rylo PG 11 | Polyglyceryl Dimer Soyate |
| | 1.00 Span 60 | Sorbitan Stearate |
| | 0.50 Vitamin E acetate | Tocopheryl Acetate |
| | 3.00 Dracorin 100 SE | Glyceryl Stearate, PEG-100 Stearate |
| | 1.00 Cremophor CO 410 | PEG-40 Hydrogenated Castor Oil |
| B | 3.00 Hybrid | |
| | 1.00 Cetiol SB 45 | Butyrospermum Parkii (Shea Butter) |
| | 6.50 Finsolv TN | C12-15 Alkyl Benzoate |
| C | 5.00 Butylene glycol | Butylene Glycol |
| | 0.30 Keltrol | Xanthan Gum |
| | 0.10 Edeta BD | Disodium EDTA |
| | 0.10 Allantoin | Allantoin |
| | 67.20 Water dem. | Aqua dem. |
| D | 2.00 Luvigel EM | Caprylic/Capric Triglyceride and |
| | | Sodium Acrylates Copolymer |

Preparation 9

| | | |
|---|---|---|
| A | 5.00 Uvinul N 539 T | Octocrylene |
| | 2.00 Parsol 1789 | Butyl Methoxydibenzoylmethane |
| | 0.80 Rylo PG 11 | Polyglyceryl Dimer Soyate |
| | 1.00 Span 60 | Sorbitan Stearate |
| | 0.50 Vitamin E acetate | Tocopheryl Acetate |
| | 3.00 Dracorin 100 SE | Glyceryl Stearate, PEG-100 Stearate |
| | 1.00 Cremophor CO 410 | PEG-40 Hydrogenated Castor Oil |
| B | 3.00 Hybrid | |
| | 1.00 Cetiol SB 45 | Butyrospermum Parkii (Shea Butter) |
| | 6.50 Finsolv TN | C12-15 Alkyl Benzoate |
| C | 5.00 Butylene glycol | Butylene Glycol |
| | 0.30 Keltrol | Xanthan Gum |
| | 0.10 Edeta BD | Disodium EDTA |
| | 0.10 Allantoin | Allantoin |
| | 67.20 Water dem. | Aqua dem. |
| D | 2.00 Luvigel EM | Caprylic/Capric Triglyceride and |
| | | Sodium Acrylates Copolymer |

Preparation 10

| | | |
|---|---|---|
| A | 5.00 Uvinul N 539 T | Octocrylene |
| | 2.00 Uvinul A Plus | Diethylamino Hydroxybenzoyl |
| | | Hexyl Benzoate |
| | 0.80 Rylo PG 11 | Polyglyceryl Dimer Soyate |
| | 1.00 Span 60 | Sorbitan Stearate |
| | 0.50 Vitamin E acetate | Tocopheryl Acetate |
| | 3.00 Dracorin 100 SE | Glyceryl Stearate, PEG-100 Stearate |
| | 1.00 Cremophor CO 410 | PEG-40 Hydrogenated Castor Oil |
| B | 3.00 Hybrid | |
| | 1.00 Cetiol SB 45 | Butyrospermum Parkii (Shea Butter) |
| | 6.50 Finsolv TN | C12-15 Alkyl Benzoate |
| C | 5.00 Butylene glycol | Butylene Glycol |
| | 0.30 Keltrol | Xanthan Gum |
| | 0.10 Edeta BD | Disodium EDTA |
| | 0.10 Allantoin | Allantoin |
| | 67.20 Water dem. | Aqua dem. |
| D | 2.00 Luvigel EM | Caprylic/Capric Triglyceride and |
| | | Sodium Acrylates Copolymer |

Preparation 11

| | | |
|---|---|---|
| A | 5.00 Uvinul N 539 T | Octocrylene |
| | 2.00 Uvinul A Plus | Diethylamino Hydroxybenzoyl |
| | | Hexyl Benzoate |
| | 0.80 Rylo PG 11 | Polyglyceryl Dimer Soyate |
| | 1.00 Span 60 | Sorbitan Stearate |
| | 0.50 Vitamin E acetate | Tocopheryl Acetate |
| | 3.00 Dracorin 100 SE | Glyceryl Stearate, PEG-100 Stearate |
| | 1.00 Cremophor CO 410 | PEG-40 Hydrogenated Castor Oil |
| B | 3.00 Hybrid | |
| | 1.00 Cetiol SB 45 | Butyrospermum Parkii (Shea Butter) |
| | 6.50 Finsolv TN | C12-15 Alkyl Benzoate |
| C | 5.00 Butylene glycol | Butylene Glycol |
| | 0.30 Keltrol | Xanthan Gum |
| | 0.10 Edeta BD | Disodium EDTA |
| | 0.10 Allantoin | Allantoin |
| | 67.20 Water dem. | Aqua dem. |
| D | 2.00 Luvigel EM | Caprylic/Capric Triglyceride and |
| | | Sodium Acrylates Copolymer |

Preparation 12

| | | |
|---|---|---|
| A | 5.00 Uvinul N 539 T | Octocrylene |
| | 2.00 Uvinul A Plus | Diethylamino Hydroxybenzoyl |
| | | Hexyl Benzoate |
| | 0.80 Rylo PG 11 | Polyglyceryl Dimer Soyate |
| | 1.00 Span 60 | Sorbitan Stearate |
| | 0.50 Vitamin E acetate | Tocopheryl Acetate |
| | 3.00 Dracorin 100 SE | Glyceryl Stearate, PEG-100 Stearate |
| | 1.00 Cremophor CO 410 | PEG-40 Hydrogenated Castor Oil |
| B | 3.00 Hybrid | |
| | 1.00 Cetiol SB 45 | Butyrospermum Parkii (Shea Butter) |
| | 6.50 Finsolv TN | C12-15 Alkyl Benzoate |
| C | 5.00 Butylene glycol | Butylene Glycol |
| | 0.30 Keltrol | Xanthan Gum |
| | 0.10 Edeta BD | Disodium EDTA |
| | 0.10 Allantoin | Allantoin |
| | 2.0 Mexoryl SX | Terephthalylidene Dicamphor |
| | | Sulfonic Acid |
| | 67.20 Water dem. | Aqua dem. |
| D | 2.00 Luvigel EM | Caprylic/Capric Triglyceride and |
| | | Sodium Acrylates Copolymer |
| | q.s. Preservative | |

Preparation 13

| | | |
|---|---|---|
| A | 5.00 Uvinul N 539 T | Octocrylene |
| | 2.00 Mexoryl SX | Terephthalylidene Dicamphor |
| | | Sulfonic Acid |
| | 0.80 Rylo PG 11 | Polyglyceryl Dimer Soyate |
| | 1.00 Span 60 | Sorbitan Stearate |
| | 0.50 Vitamin E acetate | Tocopheryl Acetate |
| | 3.00 Dracorin 100 SE | Glyceryl Stearate, PEG-100 Stearate |
| | 1.00 Cremophor CO 410 | PEG-40 Hydrogenated Castor Oil |
| B | 3.00 Hybrid | |
| | 1.00 Cetiol SB 45 | Butyrospermum Parkii (Shea Butter) |
| | 6.50 Finsolv TN | C12-15 Alkyl Benzoate |
| C | 5.00 Butylene glycol | Butylene Glycol |
| | 0.30 Keltrol | Xanthan Gum |
| | 0.10 Edeta BD | Disodium EDTA |
| | 0.10 Allantoin | Allantoin |
| | 2.0 Neoheliopan AP | |
| | 67.20 Water dem. | Aqua dem. |
| D | 2.00 Luvigel EM | Caprylic/Capric Triglyceride and |
| | | Sodium Acrylates Copolymer |

Preparation 14

| | | |
|---|---|---|
| A | 5.00 Uvinul N 539 T | Octocrylene |
| | 2.00 Uvinul A Plus | Diethylamino Hydroxybenzoyl |
| | | Hexyl Benzoate |
| | 2.00 Mexoryl XL | Drometrizole Trisiloxane |
| | 0.80 Rylo PG 11 | Polyglyceryl Dimer Soyate |
| | 1.00 Span 60 | Sorbitan Stearate |
| | 0.50 Vitamin E acetate | Tocopheryl Acetate |
| | 3.00 Dracorin 100 SE | Glyceryl Stearate, PEG-100 Stearate |
| | 1.00 Cremophor CO 410 | PEG-40 Hydrogenated Castor Oil |
| B | 3.00 Hybrid | |
| | 1.00 Cetiol SB 45 | Butyrospermum Parkii (Shea Butter) |
| | 6.50 Finsolv TN | C12-15 Alkyl Benzoate |
| C | 5.00 Butylene glycol | Butylene Glycol |
| | 0.30 Keltrol | Xanthan Gum |
| | 0.10 Edeta BD | Disodium EDTA |
| | 0.10 Allantoin | Allantoin |
| | 67.20 Water dem. | Aqua dem. |
| D | 2.00 Luvigel EM | Caprylic/Capric Triglyceride and |
| | | Sodium Acrylates Copolymer |

Preparation 15

| | | |
|---|---|---|
| A | 5.00 Uvinul N 539 T | Octocrylene |
| | 2.00 Tinosorb S | Bisethylhexyloxyphenol Methoxyphenyl |
| | | Triazine |
| | 0.80 Rylo PG 11 | Polyglyceryl Dimer Soyate |
| | 1.00 Span 60 | Sorbitan Stearate |
| | 0.50 Vitamin E acetate | Tocopheryl Acetate |
| | 3.00 Dracorin 100 SE | Glyceryl Stearate, PEG-100 Stearate |
| | 1.00 Cremophor CO 410 | PEG-40 Hydrogenated Castor Oil |
| B | 3.00 Hybrid | |
| | 1.00 Cetiol SB 45 | Butyrospermum Parkii (Shea Butter) |
| | 6.50 Finsolv TN | C12-15 Alkyl Benzoate |
| C | 5.00 Butylene glycol | Butylene Glycol |
| | 0.30 Keltrol | Xanthan Gum |
| | 0.10 Edeta BD | Disodium EDTA |
| | 0.10 Allantoin | Allantoin |
| | 67.20 Water dem. | Aqua dem. |
| D | 2.00 Luvigel EM | Caprylic/Capric Triglyceride and |
| | | Sodium Acrylates Copolymer |

Preparation 16

| | | |
|---|---|---|
| A | 2.00 Cremophor A 6 | Ceteareth-6, Stearyl Alcohol |
| | 2.00 Cremophor A 25 | Ceteareth-25 |
| | 3.00 Syncrowax HRC | Tribehenin |
| | 2.00 Lanette O | Cetearyl Alcohol |
| | 2.00 Luvitol EHO | Cetearyl Ethylhexanoate |
| | 5.00 Uvinul N539 T | Octocrylene |
| | 1.00 Ganex V-220 | VP/Eicosene Copolymer |
| | 7.00 Isopropyl palmitate | Isopropyl Palmitate |
| B | 3.00 Hybrid | |
| C | 0.50 Luvigel EM | Caprylic/Capric Triglyceride (and) |
| | | Sodium Acrylates Copolymer |
| | 63.60 Water | Aqua dem. |
| | 0.20 Edeta BD | Disodium EDTA |
| | 5.00 1,2-Propylene | Propylene Glycol |
| | glycol Care | |
| | 0.50 D-Panthenol USP | Panthenol |
| D | 1.00 Vitamin E acetate | Tocopheryl Acetate |
| | 0.50 Euxyl K 300 | Phenoxyethanol, Methylparaben, |
| | | Butylparaben, Ethylparaben, |
| | | Propylparaben, Isobutylparaben |

Preparation 17

| | | |
|---|---|---|
| A | 2.00 Cremophor A 6 | Ceteareth-6, Stearyl Alcohol |
| | 2.00 Cremophor A 25 | Ceteareth-25 |
| | 5.00 Uvinul A Plus | Diethylamino Hydroxybenzoyl |
| | | Hexyl Benzoate |
| | 2.00 Lanette O | Cetearyl Alcohol |
| | 2.00 Luvitol EHO | Cetearyl Ethylhexanoate |
| | 5.00 Uvinul MC 80 | Ethylhexyl Methoxycinnamate |
| | 1.00 Ganex V-220 | VP/Eicosene Copolymer |
| | 7.00 Isopropyl palmitate | Isopropyl Palmitate |
| B | 3.00 Hybrid | |
| C | 0.30 Carbopol Ultrez 10 | Carbomer |
| | Polymer | |
| | 0.04 Triethanolamine | Triethanolamine |
| | Care | |
| | 62.0 Water | Water |
| | 5.00 1,2-Propylene | Propylene Glycol |
| | glycol Care | |
| | 0.50 D-Panthenol USP | Panthenol |
| D | 1.00 Vitamin E acetate | Tocopheryl Acetate |
| | 0.50 Euxyl K 300 | Phenoxyethanol, Methylparaben, |
| | | Butylparaben, Ethylparaben, |
| | | Propylparaben, Isobutylparaben |

Preparation 18

| | | |
|---|---|---|
| A | 2.00 Cremophor A 6 | Ceteareth-6, Stearyl Alcohol |
| | 2.00 Cremophor A 25 | Ceteareth-25 |
| | 3.00 Syncrowax HRC | Tribehenin |
| | 2.00 Lanette O | Cetearyl Alcohol |
| | 2.00 Luvitol EHO | Cetearyl Ethylhexanoate |
| | 5.00 Uvinul N539 | Octocrylene |
| | 1.00 Ganex V-220 | VP/Eicosene Copolymer |
| | 7.00 Isopropyl palmitate | Isopropyl Palmitate |
| B | 3.00 Hybrid | |
| C | 0.50 Luvigel EM | Caprylic/Capric Triglyceride (and) |
| | | Sodium Acrylates Copolymer |
| | 64.60 Water dem. | Aqua dem. |
| | 2.00 Neoheliopan AP | Disodium Phenyl Dibenzimidazole |
| | | Tetrasulfonate |
| | 0.20 Edeta BD | Disodium EDTA |
| | 5.00 1,2-Propylene | Propylene Glycol |
| | glycol Care | |
| | 0.50 D-Panthenol USP | Panthenol |
| D | 1.00 Vitamin E acetate | Tocopheryl Acetate |
| | 0.50 Euxyl K 300 | Phenoxyethanol, Methylparaben, |
| | | Butylparaben, Ethylparaben, |
| | | Propylparaben, Isobutylparaben |

Preparation 19

| | | |
|---|---|---|
| A | 2.00 Cremophor A 6 | Ceteareth-6, Stearyl Alcohol |
| | 2.00 Cremophor A 25 | Ceteareth-25 |
| | 3.00 Syncrowax HRC | Tribehenin |
| | 2.00 Lanette O | Cetearyl Alcohol |
| | 2.00 Luvitol EHO | Cetearyl Ethylhexanoate |
| | 7.00 Uvinul N 539 T | Octocrylene |
| | 1.00 Ganex V-220 | VP/Eicosene Copolymer |
| | 7.00 Isopropyl palmitate | Isopropyl Palmitate |
| B | 3.00 Hybrid | |
| C | 0.50 Luvigel EM | Caprylic/Capric Triglyceride (and) |
| | | Sodium Acrylates Copolymer |
| | 64.60 Water dem. | Aqua dem. |
| | 1.00 Mexoryl SX | Terephthalylidene Dicamphor |
| | | Sulfonic Acid |
| | 0.20 Edeta BD | Disodium EDTA |
| | 5.00 1,2-Propylene | Propylene Glycol |
| | glycol Care | |
| | 0.50 D-Panthenol USP | Panthenol |
| D | 1.00 Vitamin E acetate | Tocopheryl Acetate |
| | 0.50 Euxyl K 300 | Phenoxyethanol, Methylparaben, |
| | | Butylparaben, Ethylparaben, |
| | | Propylparaben, Isobutylparaben |

Preparation 20

| | | |
|---|---|---|
| A | 2.00 Cremophor A 6 | Ceteareth-6, Stearyl Alcohol |
| | 2.00 Cremophor A 25 | Ceteareth-25 |
| | 3.00 Syncrowax HRC | Tribehenin |
| | 2.00 Lanette O | Cetearyl Alcohol |
| | 2.00 Luvitol EHO | Cetearyl Ethylhexanoate |
| | 2.00 Mexoryl XL | Drometrizole Trisiloxane |
| | 5.00 Uvinul N 539 T | Octocrylene |
| | 1.00 Ganex V-220 | VP/Eicosene Copolymer |
| | 7.00 Isopropyl palmitate | Isopropyl Palmitate |
| B | 3.00 Hybrid | |
| C | 0.50 Luvigel EM | Caprylic/Capric Triglyceride (and) |
| | | Sodium Acrylates Copolymer |
| | 64.60 Water dem. | Aqua dem. |
| | 0.20 Edeta BD | Disodium EDTA |
| | 5.00 1,2-Propylene | Propylene Glycol |
| | glycol Care | |
| | 0.50 D-Panthenol USP | Panthenol |
| D | 1.00 Vitamin E acetate | Tocopheryl Acetate |
| | 0.50 Euxyl K 300 | Phenoxyethanol, Methylparaben, |
| | | Butylparaben, Ethylparaben, |
| | | Propylparaben, Isobutylparaben |

Preparation 21

| | |
|---|---|
| 10.00 Hybrid | |
| 2.00 Parsol 1789 | Butyl Methoxydibenzoylmethane |
| 1.00 Uvinul T150 | Ethylhexyl Triazone |
| 2.00 Uvinul T 539 N | Octocrylene |
| 0.50 Abil 350 | Dimethicone |
| 2.75 Carnicowachs LT 20 | Carnauba (Copernica Cerifera) Wax, |
| | Paraffin |
| 3.70 Candelillawachs LT 281 LJ | Candelilla (Euphorbia Cerifera) Wax |
| 1.80 Bienenwachs 3050 PH | Bees Wax |
| 3.20 TeCero-Wachs 30445 | Microcrystalline Wax |
| 3.20 TeCero-Wachs 1030 K | Microcrystalline Wax |
| 1.34 Cutina CP | Cetyl Palmitate |
| 6.40 Vaseline | Petrolatum |
| 7.30 Softisan 100 | Hydrogenated Coco-Glycerides |
| 10.00 Luvitol EHO | Cetearyl Ethylhexanoate |
| 0.17 Bisabolol nat. | Bisabolol |
| 1.84 Vitamin E acetate | Tocopheryl Acetate |
| 0.42 D,L-alpha-Tocopherol | Tocopherol |
| 41.38 Ricinus oil | Castor (Ricinus Communis) Oil |

Preparation 22

| | |
|---|---|
| 10.00 Hybrid | |
| 2.00 Uvinul A Plus | Diethyiamino Hydroxybenzoyl |
| | Hexyl Benzoate |
| 0.50 Abil 350 | Dimethicone |
| 2.75 Carnicowachs LT 20 | Carnauba (Copernica Cerifera) Wax, |
| | Paraffin |
| 3.70 Candelillawachs LT 281 LJ | Candelilla (Euphorbia Cerifera) Wax |
| 1.80 Bienenwachs 3050 PH | Bees Wax |
| 3.20 TeCero-Wachs 30445 | Microcrystalline Wax |
| 3.20 TeCero-Wachs 1030 K | Microcrystalline Wax |
| 1.34 Cutina CP | Cetyl Palmitate |
| 6.40 Vaseline | Petrolatum |
| 7.30 Softisan 100 | Hydrogenated Coco-Glycerides |
| 10.00 Luvitol EHO | Cetearyl Ethylhexanoate |
| 0.17 Bisabolol nat. | Bisabolol |
| 1.84 Vitamin E acetate | Tocopheryl Acetate |
| 0.42 D,L-alpha-Tocopherol | Tocopherol |
| 41.38 Ricinus oil | Castor (Ricinus Communis) Oil |

Preparation 23

| | | |
|---|---|---|
| | 2.00 Cremophor A 6 | Ceteareth-6, Stearyl Alcohol |
| | 2.00 Cremophor A 25 | Ceteareth-25 |
| | 3.00 Syncrowax HRC | Tribehenin |
| | 2.00 Lanette O | Cetearyl Alcohol |
| | 2.00 Luvitol EHO | Cetearyl Ethylhexanoate |
| | 5.00 1,1-[(2,2′- | 1,1-[(2,2′-Dimethylpropoxy) |
| | Dimethylpropoxy) | carbonyl]-4,4- |
| | carbonyl]-4,4-diphenyl- | diphenyl-1,3-butadiene |
| | 1,3-butadiene | |
| | 1.00 Uvinul T 150 | Ethylhexyl Triazone |
| | 1.00 Ganex V-220 | VP/Eicosene Copolymer |
| | 7.00 Isopropyl myristate | Isopropyl Myristate |
| B | 5.00 Hybrid | |
| C | 0.20 Keltrol | Xanthan Gum |
| | 0.40 Carbopol Ultrez 10 | Carbomer |
| | Polymer | |
| | 62.10 Water dem. | Aqua dem. |
| | 0.04 Triethalonamine | Triethalonamine |
| | Care | |
| | 5.00 1,2-Propylene | Propylene Glycol |
| | glycol Care | |
| | 0.50 D-Panthenol USP | Panthenol |
| D | 1.00 Vitamin E acetate | Tocopheryl Acetate |
| | 0.50 Euxyl K 300 | Phenoxyethanol, Methylparaben, |
| | | Butylparaben, Ethylparaben, |
| | | Propylparaben, Isobutylparaben |

Preparation 24

| | | |
|---|---|---|
| A | 2.00 Luvitol EHO | Cetearyl Ethylhexanoate |
| | 5.00 Paraffin oil | Mineral Oil |
| | 5.00 Z-COTE MAX | Zink Oxide (and) |
| | | Dimethoxydiphenylsilane/ |
| | | Triethoxycaprylylsilane |
| | | Crosspolymer |
| B | 1.00 Cremophor A 6 | Ceteareth-6, Stearyl Alcohol |
| | 2.00 Cremophor A 25 | Ceteareth-25 |
| | 5.00 Hybrid | |
| | 4.00 Cetylstearyl alcohol | Cetearyl Alcohol |
| C | 0.50 D-Panthenol USP | Panthenol |
| | 5.00 1,2-Propylene | Propylene Glycol |
| | glycol Care | |
| | 7.50 Luviquat Care | Polyquaternium-44 |
| | 65.00 Water dem. | Aqua dem. |

Preparation 25

| | | |
|---|---|---|
| A | 4.00 Dehymuls SBL | Polyglyceryl-2 Dipolyhydroxystearate, |
| | | Dicaprylyl Ether, Cocoglycerides, |
| | | Sorbitan Sesquioleate, Cera Alba, |
| | | Aluminum Stearates, Dicocoyl |
| | | Pentaerythrityl Distearyl Citrate |
| | 1.00 Dehymuls PGPH | Polyglyceryl-2 Dipolyhydroxystearate |
| | 8.00 Finsolv TN | C12-15 Alkyl Benzoate |
| | 4.00 Miglyol 812 | Caprylic/Capric Triglyceride |
| | 4.00 1,1-[(2,2′- | 1,1-[(2,2′-Dimethylpropoxy) |
| | Dimethylpropoxy) | carbonyl]-4,4- |
| | carbonyl]-4,4-diphenyl- | diphenyl-1,3-butadiene |
| | 1,3-butadiene | |
| | 2.00 Uvinul N 539 T | Octocrylene |
| B | 5.00 Hybrid | |
| C | 3.00 1,2-Propylene | Propylene Glycol |
| | glycol Care | |
| | 0.30 Abiol | Imidazolidinyl Urea |
| | 1.00 Magnesium sulfate | Magnesium Sulfate |
| | 7-hydrate | |
| | ad 100 Water dem. | Aqua dem. |
| D | 0.50 Euxyl K 300 | Phenoxyethanol, Methylparaben, |
| | | Butylparaben, Ethylparaben, |
| | | Propylparaben, Isobutylparaben |

Preparation 26

| | | |
|---|---|---|
| A | 4.00 Dehymuls SBL | Polyglyceryl-2 Dipolyhydroxystearate, |
| | | Dicaprylyl Ether, Cocoglycerides, |
| | | Sorbitan Sesquioleate, Cera Alba, |
| | | Aluminum Stearates, Dicocoyl |
| | | Pentaerythrityl Distearyl Citrate |
| | 1.00 Dehymuls PGPH | Polyglyceryl-2 Dipolyhydroxystearate |
| | 8.00 Finsolv TN | C12-15 Alkyl Benzoate |
| | 4.00 Miglyol 812 | Caprylic/Capric Triglyceride |
| | 8.00 Uvinul A Plus | Diethylamino Hydroxybenzoyl |
| | | Hexyl Benzoate |
| | 2.00 Parsol 1789 | Butyl Methoxydibenzoylmethane |
| B | 5.00 Hybrid | |
| C | 3.00 1,2-Propylene | Propylene Glycol |
| | glycol Care | |
| | 0.30 Abiol | Imidazolidinyl Urea |
| | 1.00 Magnesium sulfate | Magnesium Sulfate |
| | 7-hydrate | |
| | ad 100 Water dem. | Aqua dem. |
| D | 0.50 Euxyl K 300 | Phenoxyethanol, Methylparaben, |
| | | Butylparaben, Ethylparaben, |
| | | Propylparaben, Isobutylparaben |

Preparation 27

| | | |
|---|---|---|
| A | 4.00 Dehymuls SBL | Polyglyceryl-2 Dipolyhydroxystearate, |
| | | Dicaprylyl Ether, Cocoglycerides, |
| | | Sorbitan Sesquioleate, Cera Alba, |
| | | Aluminum Stearates, Dicocoyl |
| | | Pentaerythrityl Distearyl Citrate |
| | 1.00 Dehymuls PGPH | Polyglyceryl-2 Dipolyhydroxystearate |
| | 8.00 Finsolv TN | C12-15 Alkyl Benzoate |
| | 4.00 Miglyol 812 | Caprylic/Capric Triglyceride |
| | 8.00 Uvinul A Plus | Diethylamino Hydroxybenzoyl |
| | | Hexyl Benzoate |
| | 2.00 Tinosorb M | Methylene Bisbenzotriazolyl |
| | | Tetramethylbutylphenol |
| B | 5.00 Hybrid | |
| C | 3.00 1,2-Propylene | Propylene Glycol |
| | glycol Care | |
| | 0.30 Abiol | Imidazolidinyl Urea |
| | 1.00 Magnesium sulfate | Magnesium Sulfate |
| | 7-hydrate | |
| | ad 100 Water dem. | Aqua dem. |
| D | 0.50 Euxyl K 300 | Phenoxyethanol, Methylparaben, |
| | | Butylparaben, Ethylparaben, |
| | | Propylparaben, Isobutylparaben |

Preparation 28

| | | |
|---|---|---|
| A | 4.00 Dehymuls SBL | Polyglyceryl-2 Dipolyhydroxystearate, |
| | | Dicaprylyl Ether, Cocoglycerides, |
| | | Sorbitan Sesquioleate, Cera Alba, |
| | | Aluminum Stearates, Dicocoyl |
| | | Pentaerythrityl Distearyl Citrate |
| | 1.00 Dehymuls PGPH | Polyglyceryl-2 Dipolyhydroxystearate |
| | 8.00 Finsolv TN | C12-15 Alkyl Benzoate |
| | 4.00 Miglyol 812 | Caprylic/Capric Triglyceride |
| | 7.00 Uvinul N539 T | Octocrylene |
| | 2.00 Mexoryl XL | Drometrizole Disiloxane |
| B | 5.00 Hybrid | |
| C | 3.00 1,2-Propylene | Propylene Glycol |
| | glycol Care | |
| | 0.30 Abiol | Imidazolidinyl Urea |
| | 1.00 Magnesium sulfate | Magnesium Sulfate |
| | 7-hydrate | |
| | ad 100 Water dem. | Aqua dem. |
| D | 0.50 Euxyl K 300 | Phenoxyethanol, Methylparaben, |
| | | Butylparaben, Ethylparaben, |
| | | Propylparaben, Isobutylparaben |

Preparation 29

| | | |
|---|---|---|
| A | 5.00 Uvinul A Plus | Diethylamino Hydroxybenzoyl |
| | | Hexyl Benzoate |
| | 2.00 Hybrid | |
| | 10.00 Miglyol 812 | Caprylic/Capric Triglyceride |
| | 1.50 Dow Corning 350 Fluid | Dimethicone |
| | 3.00 Finsolv TN | C12-15 Alkyl Benzoate |
| | 1.00 Cremophor CO 40 | PEG-40 Hydrogenated Castor Oil |
| B | 2.00 Luvigel EM | Caprylic/Capric Triglyceride, |
| | | Sodium Acrylates Copolymer |
| C | 54.80 Water dem. | Aqua dem. |
| D | 2% Dihydroxacetone | Dihydroxacetone |
| | 5.00 1,2-Propylene | Propylene Glycol |
| | glycol Care | |
| | 0.50 Cremophor A 25 | Ceteareth-25 |
| | 1.00 D-Panthenol 50 P | Panthenol, Propylene Glycol |
| | 1.00 Vitamin E acetate | Tocopheryl Acetate |
| | 0.20 Bisabolol rac. | Bisabolol |

Preparation 30

| | | |
|---|---|---|
| A | 4.00 Dehymuls SBL | Polyglyceryl-2 Dipolyhydroxystearate, |
| | | Dicaprylyl Ether, Cocoglycerides, |
| | | Sorbitan Sesquioleate, Cera Alba, |
| | | Aluminum Stearates, Dicocoyl |
| | | Pentaerythrityl Distearyl Citrate |
| | 1.00 Dehymuls PGPH | Polyglyceryl-2 Dipolyhydroxystearate |
| | 6.00 Finsolv TN | C12-15 Alkyl Benzoate |
| | 6.00 Miglyol 812 | Caprylic/Capric Triglyceride |
| | 2.00 Uvinul A Plus | Diethylamino Hydroxybenzoyl |
| | | Hexyl Benzoate |
| | 3.00 Uvinul M 40 | Benzophenone-3 |
| B | 5.00 Hybrid | |
| C | 3.00 1,2-Propylene | Propylene Glycol |
| | glycol Care | |
| | 0.30 Chemag 2000 | Imidazolidinyl Urea |
| | 1.00 Magnesium sulfate- | Magnesium Sulfate |
| | 7-hydrate | |
| | 65.20 Water dem. | Aqua dem. |
| D | 0.50 Euxyl K 300 | Phenoxyethanol, Methylparaben, |
| | | Butylparaben, Ethylparaben, |
| | | Propylparaben, Isobutylparaben |

Preparation 31

| | | |
|---|---|---|
| A | 4.00 Tego Care 450 | Polyglyceryl-3 Methyl Glucose |
| | | Distearate |
| | 4.00 Uvinul MC 80 | Ethylhexyl Methoxycinnamate |
| | 3.00 Uvinul N 539 T | Octocrylene |
| | 2.00 Uvinul T 150 | Ethylhexyl Triazone |
| | 0.50 Vitamin E acetate | Tocopheryl Acetate |
| B | 6.00 Isohexadecane | Isohexadecane |
| | 4.00 Cosmacol EMI | Di-C12-13 Alkyl Malate |
| | 3.50 Cetiol SN | Cetearyl Isononanoate |
| | 1.00 Ganex V-220 | VP/Eicosene Copolymer |
| | 5.00 Hybrid | |
| C | 60.66 Water dem. | Water |
| | 5.00 Glycerin 87% | Glycerin |
| | 1.00 Lanette E | Sodium Cetearyl Sulfate |
| | 0.30 Carbopol Ultrez 10 | Carbomer |
| | Polymer | |
| | 0.04 Triethanolamine | Triethanolamine |
| | Care | |

Preparation 32

| | | |
|---|---|---|
| A | 4.00 Tego Care 450 | Polyglyceryl-3 Methyl Glucose |
| | | Distearate |
| | 4.50 Uvinul A Plus | Diethylamino Hydroxybenzoyl |
| | | Hexyl Benzoate |
| | 3.00 Uvinul N 539 T | Octocrylene |
| | 2.00 Neoheliopan HMS | Homosalate |
| | 0.50 Vitamin E acetate | Tocopheryl Acetate |
| B | 5.00 Isohexadecane | Isohexadecane |
| | 5.00 Cosmacol EMI | Di-C12-13 Alkyl Malate |
| | 3.50 Cetiol SN | Cetearyl Isononanoate |
| | 1.00 Ganex V-220 | VP/Eicosene Copolymer |
| | 5.00 Hybrid | |
| C | 60.66 Water dem. | Aqua dem. |
| | 5.00 Glycerin 87% | Glycerin |
| | 1.00 Lanette E | Sodium Cetearyl Sulfate |
| | 0.30 Carbopol Ultrez 10 | Carbomer |
| | Polymer | |
| | 0.04 Triethanolamine | Triethanolamine |
| | Care | |

Preparation 33

| | | |
|---|---|---|
| A | 4.00 Tego Care 450 | Polyglyceryl-3 Methyl Glucose |
| | | Distearate |
| | 4.50 Uvinul N539T | Ethylhexyl Methoxycinnamate |
| | 3.00 Neoheliopan OS | Octisalate |
| | 2.00 Neoheliopan HMS | Homosalate |
| | 0.50 Vitamin E acetate | Tocopheryl Acetate |
| B | 5.00 Isohexadecane | Isohexadecane |
| | 5.00 Cosmacol EMI | Di-C12-13 Alkyl Malate |
| | 3.50 Cetiol SN | Cetearyl Isononanoate |
| | 1.00 Ganex V-220 | VP/Eicosene Copolymer |
| | 5.00 Hybrid | |
| C | 60.20 Water dem. | Aqua dem. |
| | 5.00 Glycerin 87% | Glycerin |
| | 1.00 Lanette E | Sodium Cetearyl Sulfate |
| | 0.30 Keltrol | Xanthan Gum |

Preparation 34

| | | |
|---|---|---|
| A | 4.00 Tego Care 450 | Polyglyceryl-3 Methyl Glucose |
| | | Distearate |
| | 3.00 Uvinul N 539 T | Octocrylene |
| | 3.00 Neoheliopan OS | Octisalate |
| | 2.00 Neoheliopan HMS | Homosalate |
| | 0.50 Vitamin E acetate | Tocopheryl Acetate |
| B | 5.00 Isohexadecane | Isohexadecane |
| | 5.00 Cosmacol EMI | Di-C12-13 Alkyl Malate |
| | 3.50 Cetiol SN | Cetearyl Isononanoate |
| | 1.00 Ganex V-220 | VP/Eicosene Copolymer |
| | 3.00 Hybrid | |
| | 4.00 Z-COTE MAX | Zinc Oxide (and) |
| | | Dimethoxydiphenylsilane/ |
| | | Triethoxycaprylylsilane |
| | | Crosspolymer |
| C | 60.20 Water dem. | Aqua dem. |
| | 5.00 Glycerin 87% | Glycerin |
| | 1.00 Lanette E | Sodium Cetearyl Sulfate |
| | 0.80 Luvigel EM | Caprylic/Capric triglyceride and |
| | | Sodium Acrylates Copolymer |

Preparation 35

| | | |
|---|---|---|
| A | 5.00 Cosmacol EMI | Di-C12-13 Alkyl Malate |
| | 7.50 Uvinul N 539 T | Octocrylene |
| | 2.00 Mexoryl XL | Drometrizole Disiloxane |
| B | 4.00 Tego Care 450 | Polyglyceryl-3 Methyl Glucose |
| | | Distearate |
| | 5.00 Isohexadecane | Isohexadecane |
| | 3.50 Cetiol SN | Cetearyl Isononanoate |
| | 0.50 Vitamin E acetate | Tocopheryl Acetate |
| | 1.00 Ganex V-220 | VP/Eicosene Copolymer |
| | 5.00 Hybrid | |
| C | 5.00 Glycerin 87% | Glycerin |
| | 2.00 Lanette E | Sodium Cetearyl Sulfate |
| | 0.30 Carbopol Ultrez 10 | Carbomer |
| | Polymer | |
| | 1.00 Pationic 138 C | Sodium Lauroyl Lactylate |
| | 1.00 Pationic SSL | Sodium Stearoyl Lactylate |
| | 41.50 Water dem. | Aqua dem. |
| D | 5.00 Eusolex 232 | Phenylbenzimidazole Sulfonic Acid |
| | 10.00 Water dem. | Aqua dem. |
| E | 1.0 Sodium hydroxide | Sodium Hydroxide |
| F | 1.00 Phenonip | Phenoxyethanol, Methylparaben, |
| | | Ethylparaben, Butylparaben, |
| | | Propylparaben, Isobutylparaben |

Preparation 36

| | | |
|---|---|---|
| A | 5.00 Cosmacol EMI | Di-C12-13 Alkyl Malate |
| | 5.50 Uvinul N 539 T | Octocrylene |
| | 2.00 Uvinul A Plus | Diethylamino Hydroxybenzoyl |
| | | Hexyl Benzoate |
| | 2.00 Mexoryl XL | Drometrizole Disiloxane |
| B | 4.00 Tego Care 450 | Polyglyceryl-3 Methyl Glucose |
| | | Distearate |
| | 5.00 Isohexadecane | Isohexadecane |
| | 3.50 Cetiol SN | Cetearyl Isononanoate |
| | 0.50 Vitamin E acetate | Tocopheryl Acetate |
| | 1.00 Ganex V-220 | VP/Eicosene Copolymer |
| | 5.00 Hybrid | |
| C | 5.00 Glycerin 87% | Glycerin |
| | 2.00 Lanette E | Sodium Cetearyl Sulfate |
| | 0.30 Keltrol | Xanthan Gum |
| | 1.00 Pationic 138 C | Sodium Lauroyl Lactylate |
| | 1.00 Pationic SSL | Sodium Stearoyl Lactylate |
| | 41.50 Water dem. | Aqua dem |
| D | 5.00 Eusolex 232 | Phenylbenzimidazole Sulfonic Acid |
| | 10.00 Water dem. | Aqua dem. |
| E | 0.70 Sodium hydroxide | Sodium Hydroxide |
| F | 1.00 Phenonip | Phenoxyethanol, Methylparaben, |
| | | Ethylparaben, Butylparaben, |
| | | Propylparaben, Isobutylparaben |

Preparation 37

| | | |
|---|---|---|
| A | 5.00 Cosmacol EMI | Di-C12-13 Alkyl Malate |
| | 5.50 Uvinul N 539 T | Octocrylene |
| | 2.00 1,1-[(2,2′- | 1,1-[(2,2′-Dimethylpropoxy) |
| | Dimethylpropoxy | carbonyl]-4,4- |
| | carbonyl]-4,4-diphenyl- | diphenyl-1,3-butadiene |
| | 1,3-butadiene | |
| | 2.00 Mexoryl XL | Drometrizole Disiloxane |
| B | 4.00 Tego Care 450 | Polyglyceryl-3 Methyl Glucose |
| | | Distearate |
| | 5.00 Isohexadecane | Isohexadecane |
| | 3.50 Cetiol SN | Cetearyl Isononanoate |
| | 0.50 Vitamin E acetate | Tocopheryl Acetate |
| | 1.00 Ganex V-220 | VP/Eicosene Copolymer |
| | 5.00 Hybrid | |
| C | 5.00 Glycerin 87% | Glycerin |
| | 2.00 Lanette E | Sodium Cetearyl Sulfate |
| | 0.30 Keltrol | Xanthan Gum |
| | 1.00 Pationic 138 C | Sodium Lauroyl Lactylate |
| | 1.00 Pationic SSL | Sodium Stearoyl Lactylate |
| | 41.50 Water dem. | Aqua dem. |
| D | 5.00 Eusolex 232 | Phenylbenzimidazole Sulfonic Acid |
| | 10.00 Water dem. | Aqua dem. |
| E | 0.70 Sodium hydroxide | Sodium Hydroxide |
| F | 1.00 Phenonip | Phenoxyethanol, Methylparaben, |
| | | Ethylparaben, Butylparaben, |
| | | Propylparaben, Isobutylparaben |

Preparation 38

| | | |
|---|---|---|
| A | 3.00 Glycerin 87% | Glycerin |
| | 0.20 Edeta BD | Disodium EDTA |
| | 0.30 Abiol | Imidazolidinyl Urea |
| | 1.00 Plantacare 2000 | Decyl Glucoside |
| | 0.30 Keltrol T | Xanthan Gum |
| | 2.00 D-Panthenol 50 P | Panthenol, Propylene Glycol |
| | 58.00 Water dem. | Aqua dem. |
| B | 5.00 Hybrid | |
| C | 1.50 Uvasorb HEB | Diethylhexyl Butamido Triazone |
| | 5.00 Uvinul A Plus | Diethylamino Hydroxybenzoyl |
| | | Hexyl Benzoate |
| | 2.00 Dow Corning 345 Fluid | Cyclopentasiloxane, |
| | | Cyclohexasiloxane |
| | 3.50 Cremophor A 6 | Ceteareth-6, Stearyl Alcohol |
| | 1.50 Cremophor A 25 | Ceteareth-25 |
| | 0.50 Bienenwachs 3044 PH | Bees Wax |
| | 3.00 Lanette O | Cetearyl Alcohol |
| | 10.00 Miglyol 812 | Caprylic/Capric Triglyceride |
| D | 1.00 Vitamin E acetate | Tocopheryl Acetate |
| | 0.20 Bisabolol rac. | Bisabolol |

Preparation 39

| | | |
|---|---|---|
| A | 3.00 Glycerin 87% | Glycerin |
| | 0.20 Edeta BD | Disodium EDTA |
| | 0.30 Abiol | Imidazolidinyl Urea |
| | 1.00 Plantacare 2000 | Decyl Glucoside |
| | 0.30 Keltrol T | Xanthan Gum |
| | 2.00 D-Panthenol 50 P | Panthenol, Propylene Glycol |
| | 57.00 Water dem. | Aqua dem. |
| B | 5.00 Hybrid | |
| C | 7.50 Uvinul Plus | Diethylamino Hydroxybenzoyl |
| | | Hexyl Benzoate |
| | 2.00 Uvinul N 539 T | Octocrylene |
| | 2.00 Dow Corning 345 Fluid | Cyclopentasiloxane, |
| | | Cyclohexasiloxane |
| | 3.50 Cremophor A 6 | Ceteareth-6, Stearyl Alcohol |
| | 1.50 Cremophor A 25 | Ceteareth-25 |
| | 0.60 Bienenwachs 3044 PH | Bees Wax |
| | 3.00 Lanette O | Cetearyl Alcohol |
| | 10.00 Miglyol 812 | Capryiic/Capric Triglyceride |
| D | 1.00 Vitamin E acetate | Tocopheryl Acetate |
| | 0.20 Bisabolol rac. | Bisabolol |

Preparation 40

| | | |
|---|---|---|
| A | 3.00 Glycerin 87% | Glycerin |
| | 0.20 Edeta BD | Disodium EDTA |
| | 0.30 Abiol | Imidazolidinyl Urea |
| | 1.00 Plantacare 2000 | Decyl Glucoside |
| | 0.30 Keltrol T | Xanthan Gum |
| | 2.00 Mexoryl SL | Benzylidene Camphor Sulfonic Acid |
| | 58.50 Water dem. | Aqua dem. |
| B | 6.00 Hybrid | |
| | 6.00 Uvinul N 539 T | Octocrylene |
| | 2.00 Parsol 1789 | Butyl Methoxydibenzoylmethane |
| | 2.00 Dow Corning 345 Fluid | Cyclopentasiloxane, |
| | | Cyclohexasiloxane |
| | 3.50 Cremophor A 6 | Ceteareth-6, Stearyl Alcohol |
| | 1.50 Cremophor A 25 | Ceteareth-25 |
| | 0.50 Bienenwachs 3044 PH | Bees Wax |
| | 3.00 Lanette O | Cetearyl Alcohol |
| | 10.00 Miglyol 812 | Caprylic/Capric Triglyceride |
| D | 1.00 Vitamin E acetate | Tocopheryl Acetate |
| | 0.20 Bisabolol rac. | Bisabolol |

Preparation 41

| | | |
|---|---|---|
| A | 2.00 Glycerin 87% | Glycerin |
| | 1.00 Dihydroxyacetone | Dihydroxyacetone |
| | 0.20 Edeta BD | Disodium EDTA |
| | 0.30 Abiol | Imidazolidinyl Urea |
| | 1.00 Plantacare 2000 | Decyl Glucoside |
| | 0.30 Keltrol T | Xanthan Gum |
| | 2.00 Mexoryl SL | Benzylidene Camphor Sulfonic Acid |
| | 58.50 Water dem. | Aqua dem. |
| B | 6.00 Hybrid | |
| | 6.00 Uvinul N 539 T | Octocrylene |
| | 2.00 Tinosorb M | Methylene Bisbenzotriazolyl |
| | | Tetramethylbutylphenol |
| | 2.00 Dow Corning 345 Fluid | Cyclopentasiloxane, |
| | | Cyclohexasiloxane |
| | 3.50 Cremophor A 6 | Ceteareth-6, Stearyl Alcohol |
| | 1.50 Cremophor A 25 | Ceteareth-25 |
| | 0.50 Bienenwachs 3044 PH | Bees Wax |
| | 3.00 Lanette O | Cetearyl Alcohol |
| | 10.00 Miglyol 812 | Caprylic/Capric Triglyceride |
| D | 1.00 Vitamin E acetate | Tocopheryl Acetate |
| | 0.20 Bisabolol rac. | Bisabolol |

Preparation 42

| | | |
|---|---|---|
| A | 3.00 Glycerin 87% | Glycerin |
| | 0.20 Edeta BD | Disodium EDTA |
| | 0.30 Abiol | Imidazolidinyl Urea |
| | 1.00 Plantacare 2000 | Decyl Glucoside |
| | 0.30 Keltrol T | Xanthan Gum |
| | 2.00 Neoheliopan AP | Disodium Phenyl Dibenzimidazole |
| | | Tetrasulfonate |
| | 58.50 Water dem. | Aqua dem. |
| B | 6.00 Hybrid | |
| | 6.00 Uvinul N 539 T | Octocrylene |
| | 2.00 Parsol 1789 | Butyl Methoxydibenzoylmethane |
| | 2.00 Dow Corning 345 Fluid | Cyclopentasiloxane, |
| | | Cyclohexasiloxane |
| | 3.50 Cremophor A 6 | Ceteareth-6, Stearyl Alcohol |
| | 1.50 Cremophor A 25 | Ceteareth-25 |
| | 0.50 Bienenwachs 3044 PH | Bees Wax |
| | 3.00 Lanette O | Cetearyl Alcohol |
| | 10.00 Miglyol 812 | Caprylic/Capric Triglyceride |
| D | 1.00 Vitamin E acetate | Tocopheryl Acetate |
| | 0.20 Bisabolol rac. | Bisabolol |

Preparation 43

| | | |
|---|---|---|
| A | 25.00 Dow Corning 345 Fluid | Cyclopentasiloxane, |
| | | Cyclohexasiloxane |
| | 20.00 Dow Corning 245 Fluid | Cyclopentasiloxane |
| | 8.00 Uvinul N539T | Octocrylene |
| | 4.00 Abil EM 90 | Cetyl PEG/PPG-10/1 Dimethicone |
| | 7.00 Z-COTE MAX | Zinc Oxide (and) |
| | | Dimethoxydiphenylsilane/ |
| | | Trielhoxycaprylylsilane |
| | | Crosspolymer |
| B | 17.00 Ethanol 95% | Alcohol |
| | 10.00 Hybrid | |
| | 5.00 Water dem. | Aqua dem. |
| | 3.00 Glycerin 87% | Glycerin |
| | 1.00 Talc | Talc |

Preparation 43

| | | |
|---|---|---|
| A | 2.00 Cremophor WO 7 | PEG-7 Hydrogenated Castor Oil |
| | 6.00 Abil B 8839 | Cyclopentasiloxane, |
| | | Cyclohexasiloxane |
| | 3.00 Hybrid | |
| | 4.00 Isopropyl palmitate | Isopropyl Palmitate |
| | 1.00 Abil 350 | Dimethicone |
| B | 5.00 Uvinul N539 T | Octocrylene |
| | 1.50 Uvinul A Plus | Diethylamino Hydroxybenzoyl |
| | | Hexyl Benzoate |
| | 1.00 Uvasorb HEB | Diethylhexyl Butamido Triazone |
| | 7.00 Finsolv TN | C12-15 Alkyl Benzoate |
| | 4.00 Abil WE 09 | Polyglyceryl-4 Isostearate, Cetyl |
| | | PEG/PPG-10/1 Dimethicone, Hexyl |
| | | Laurate |
| C | 0.50 Sodium chloride | Sodium Chloride |
| | 0.20 Edeta BD | Disodium EDTA |
| | 62.30 Water dem. | Aqua dem. |
| D | 1.00 Vitamin E acetate | Tocopheryl Acetate |
| | 1.00 Phenonip | Phenoxyethanol, Methylparaben, |
| | | Ethylparaben, Butylparaben, |
| | | Propylparaben, Isobutylparaben |

Preparation 44

| | | |
|---|---|---|
| A | 2.00 Cremophor WO 7 | PEG-7 Hydrogenated Castor Oil |
| | 6.00 Abil B 8839 | Cyclopentasiloxane, |
| | | Cyclohexasiloxane |
| | 3.00 Hybrid | |
| | 4.00 Isopropyl palmitate | Isopropyl Palmitate |
| | 1.00 Abil 350 | Dimethicone |
| B | 4.00 Uvinul N539 T | Octocrylene |
| | 3.0 Uvinul A Plus | Diethylamino Hydroxybenzoyl |
| | | Hexyl Benzoate |
| | 1.00 Mexoryl SL | Drometrizole Trisiloxane |
| | 7.00 Finsolv TN | C12-15 Alkyl Benzoate |
| | 4.00 Abil WE 09 | Polyglyceryl-4 Isostearate, Cetyl |
| | | PEG/PPG-10/1 Dimethicone, Hexyl |
| | | Laurate |
| C | 0.50 Sodium chloride | Sodium Chloride |
| | 0.20 Edeta BD | Disodium EDTA |
| | 62.30 Water dem. | Aqua dem. |
| D | 1.00 Vitamin E acetate | Tocopheryl Acetate |
| | 1.00 Phenonip | Phenoxyethanol, Methylparaben, |
| | | Ethylparaben, Butylparaben, |
| | | Propylparaben, Isobutylparaben |

Preparation 45

| | | |
|---|---|---|
| A | 2.00 Cremophor WO 7 | PEG-7 Hydrogenated Castor Oil |
| | 6.00 Abil B 8839 | Cyclopentasiloxane, |
| | | Cyclohexasiloxane |
| | 5.00 Hybrid | |
| | 4.00 Isopropyl palmitate | Isopropyl Palmitate |
| | 1.00 Abil 350 | Dimethicone |
| B | 3.00 Uvinul N 539 T | Octocrylene |
| | 1.00 Mexoryl XL | Drometrizole Trisiloxane |
| | 1.00 Tinosorb S | Bisethylhexyloxyphenol |
| | | Methoxyphenyl Triazine |
| | 1.00 1,1-(2,2′- | 1,1-[(2,2′-Dimethylpropoxy) |
| | Dimethylpropoxy) | carbonyl]-4,4- |
| | carbonyl]-4,4-diphenyl- | diphenyl-1,3-butadiene |
| | 1,3-butadiene | |
| | 7.00 Finsolv TN | C12-15 Alkyl Benzoate |
| | 4.00 Abil WE 09 | Polyglyceryl-4 Isostearate, Cetyl |
| | | PEG/PPG-10/1 Dimethicone, Hexyl |
| | | Laurate |
| C | 0.50 Sodium chloride | Sodium Chloride |
| | 0.20 Edeta BD | Disodium EDTA |
| | 62.30 Water dem. | Aqua dem. |
| D | 1.00 Vitamin E acetate | Tocopheryl Acetate |
| | 1.00 Phenonip | Phenoxyethanol, Methylparaben, |
| | | Ethylparaben, Butylparaben, |
| | | Propylparaben, Isobutylparaben |

Preparation 46

| | | |
|---|---|---|
| A | 4.50 Uvinul MC 80 | Ethylhexyl Methoxycinnamate |
| | 3.00 Uvinul N 539 T | Octocrylene |
| | 1.00 Tinosorb S | Bisethylhexyloxyphenol |
| | | Methoxyphenyl Triazine |
| | 2.50 Cosmacol EMI | Di-C12-13 Alkyl Malate |
| B | 0.50 Vitamin E acetate | Tocopheryl Acetate |
| | 4.00 Tego Care 450 | Polyglyceryl-3 Methyl Glucose |
| | | Distearate |
| C | 3.50 Cetiol SN | Cetearyl Isononanoate |
| | 1.00 Ganex V-220 | VP/Eicosene Copolymer |
| | 5.00 Isohexadecane | Isohexadecane |
| | 2.50 Cosmacol EMI | Di-C12-13 Alkyl Malate |
| | 5.00 Hybrid | |
| D | 45.00 Water dem. | Aqua dem. |
| | 5.00 Glycerin 87% | Glycerin |
| | 1.00 Lanette E | Sodium Cetearyl Sulfate |
| | 1.00 Dihydroxyacetone | Dihydroxyacetone |
| | 0.50 Keltrol | Xanthan Gum |
| E | 1.00 Phenonip | Phenoxyethanol, Methylparaben, |
| | | Ethylparaben, Butylparaben, |
| | | Propylparaben, Isobutylparaben |

Preparation 47

| | | |
|---|---|---|
| A | 1.50 Mexoryl XL | Drometrizole Trisiloxane |
| | 5.00 Uvinul N 539 T | Octocrylene |
| | 1.00 Tinosorb S | Bisethylhexyloxyphenol |
| | | Methoxyphenyl Triazine |
| | 2.50 Cosmacol EMI | Di-C12-13 Alkyl Malate |
| B | 0.50 Vitamin E acetate | Tocopheryl Acetate |
| | 4.00 Tego Care 450 | Polyglyceryl-3 Methyl Glucose |
| | | Distearate |
| C | 3.50 Cetiol SN | Cetearyl Isononanoate |
| | 1.00 Ganex V-220 | VP/Eicosene Copolymer |
| | 5.00 Isohexadecane | Isohexadecane |
| | 2.50 Cosmacol EMI | Di-C12-13 Alkyl Malate |
| | 3.00 Hybrid | |
| D | 46.00 Water dem. | Aqua dem. |
| | 5.00 Glycerin 87% | Glycerin |
| | 1.00 Lanette E | Sodium Cetearyl Sulfate |
| | 2.00 Dihydroxyacetone | Dihydroxyacetone |
| | 15.00 Luviquat Care | Polyquaternium-44 |
| | 0.50 Keltrol | Xanthan Gum |
| E | 1.00 Phenonip | Phenoxyethanol, Methylparaben, |
| | | Ethylparaben, Butylparaben, |
| | | Propylparaben, Isobutylparaben |
| A | 1.00 Abil Care 85 | Bis-PEG/PPG-16/16 PEG/PPG-16/16 |
| | | Dimethicone, Caprylic/Capric |
| | | Triglyceride |
| | 3.00 Cremophor CO 40 | PEG-40 Hydrogenated Castor Oil |
| | 0.30 Cremophor WO 7 | PEG-7 Hydrogenated Castor Oil |
| | 5.00 Uvinul A Plus | Diethylamino Hydroxybenzoyl |
| | | Hexyl Benzoate |
| | 10.00 Witconol APM | PPG-3 Myristyl Ether |
| | 2.00 Uvinul T 150 | Ethylhexyl Triazone |
| | 1.00 Dow Corning 345 Fluid | Cyclopentasiloxane, |
| | | Cyclohexasiloxane |
| | 5.00 Uvinul N 539 T | Octocrylene |
| B | 5.00 Hybrid | |
| C | 5.00 1,2-Propylene glycol | Propylene Glycol |
| | 2.00 D-Panthenol 50 P | Panthenol, Propylene Glycol |
| | 0.20 Keltrol | Xanthan Gum |
| | 0.10 Edeta BD | Disodium EDTA |
| | 1.50 Simulgel 600 | Acrylamide/Sodium |
| | | Acryloyldimethyltaurate |
| | | Copolymer, Isohexadecane, |
| | | Polysorbate 80 |
| | 58.40 Water dem. | Aqua dem. |
| D | 0.50 Glydant | DMDM Hydantoin |

Preparation 48

| | | |
|---|---|---|
| A | 10.00 Uvinul A Plus | Diethylamino Hydroxybenzoyl |
| | | Hexyl Benzoate |
| | 3.00 Uvinul N 539 T | Octocrylene |
| | 4.00 Eumulgin VL 75 | Lauryl Glucoside, Polyglyceryl-2 |
| | | Dipolyhydroxystearate, Glycerin |
| | 8.00 Cetiol B | Dibutyl Adipate |
| | 8.00 Finsolv TN | C12-15 Alkyl Benzoate |
| | 12.00 Myritol 331 | Cocoglycerides |
| | 1.00 Lanette E | Sodium Cetearyl Sulfate |
| | 2.00 Lanette O | Cetearyl Alcohol |
| B | 3.00 Hybrid | |
| C | 35.08 Water dem. | Water |
| | 0.38 Citric acid | Citric Acid |
| | 3.00 Glycerin 87% | Glycerin |
| | 0.05 Edeta BD | Disodium EDTA |
| | 0.20 Dihydroxyacetone | Dihydroxyacetone |
| | 0.30 Carbopol Ultrez 10 | Carbomer |
| | Polymer | |
| | 0.04 Triethanolamine | Triethanolamine |
| | Care | |
| D | 0.50 Phenonip | Phenoxyethanol, Methylparaben, |
| | | Ethylparaben, Butylparaben, |
| | | Propylparaben, Isobutylparaben |
| | 1.00 Vitamin E acetate | Tocopheryl Acetate |

Preparation 49

| | | |
|---|---|---|
| A | 9.00 Uvinul A Plus | Diethylamino Hydroxybenzoyl |
| | | Hexyl Benzoate |
| | 4.00 Uvinul N 539 T | Octocrylene |
| | 2.00 1,1-[(2,2′- | 1,1-[(2,2′-Dimethylpropoxy) |
| | Dimethylpropoxy)- | carbonyl]-4,4- |
| | carbonyl]-4,4-diphenyl- | diphenyl-1,3-butadiene |
| | 1,3-butadiene | |
| | 1.00 UvasorbHEB | Diethylhexyl Butamido Triazone |
| | 4.00 Eumulgin VL 75 | Lauryl Glucoside, Polyglyceryl-2 |
| | | Dipolyhydroxystearate, Glycerin |
| | 8.00 Cetiol B | Dibutyl Adipate |
| | 8.00 Finsolv TN | C12-15 Alkyl Benzoate |
| | 12.00 Myritol 331 | Cocoglycerides |
| | 1.00 Lanette E | Sodium Cetearyl Sulfate |
| | 2.00 Lanette O | Cetearyl Alcohol |
| B | 3.00 Hybrid | |
| C | 40.0 Water dem. | Aqua dem. |
| | 0.40 Citric acid | Citric Acid |
| | 3.00 Glycerin 87% | Glycerin |
| | 0.05 Edeta BD | Disodium EDTA |
| | 0.50 Dihydroxyacetone | Dihydroxyacetone |
| | 0.30 Ultrez 10 Polymer | Carbomer |
| | 0.04 Triethanolamine | Triethanolamine |
| | Care | |
| D | 0.50 Phenonip | Phenoxyethanol, Methylparaben, |
| | | Ethylparaben, Butylparaben, |
| | | Propylparaben, Isobutylparaben |
| | 1.00 Vitamin E acetate | Tocopheryl Acetate |

1-6. (canceled)

7. A photoprotective preparation comprising
nanoparticles of at least one metal derivative
at least one structural unit chemically bonded to the nanoparticles of at least one metal derivative, selected from the group consisting of derivatives of triazine, derivatives of hydroxybenzophenone and derivatives of 4,4′diarylbutadiene.

8. The photoprotective preparation according to claim 1, comprising at least one further organic UV photoprotective agent different from the UV photoprotective agent bonded to the nanoparticles.

9. A photoprotective preparation comprising
nanoparticles of at least one metal derivative,
at least one organic UV photoprotective agent chemically bonded to the nanoparticles of at least one metal derivative and
at least one further organic UV photoprotective agent which is different from the UV photoprotective agent bonded to the nanoparticles and different from octyl methoxycinnamate.

10. The photoprotective preparation according to claim 2, where the at least one further organic UV photoprotective agent is selected from the group consisting of 2-ethylhexyl 2-cyano-3,3-diphenylacrylate (CAS No. 6197-30-4), ethylhexyl salicylate (CAS No. 118-60-5), homosalate (CAS No. 118-56-9), dimethicodiethyl benzalmalonate (CAS No. 207574-74-1) and mixtures thereof.

11. The photoprotective preparation according to claim 1, further comprising dihydroxyacetone.

12. The photoprotective preparation according to claim 1, where the metal derivative is selected from the derivatives of titanium and/or zinc.
